(19) United States
(12) Patent Application Publication
(10) Pub. No.: US 2024/0374201 A1
(43) Pub. Date: Nov. 14, 2024

(54) DERIVATION OF ELECTRODERMAL ACTIVITY RESPONSE FROM AN ELECTROCARDIOGRAM SIGNAL

(71) Applicant: University of Connecticut, Farmington, CT (US)

(72) Inventors: Ki Chon, Portsmouth, RI (US); Farnoush Baghestanibarzoki, Chaplin, CT (US); Youngsun Kong, Vernon, CT (US)

(21) Appl. No.: 18/658,442

(22) Filed: May 8, 2024

Related U.S. Application Data
(60) Provisional application No. 63/500,810, filed on May 8, 2023.

(51) Int. Cl. A61B 5/00; A61B 5/0533

(52) U.S. Cl. CPC A61B 5/4035; A61B 5/0533; A61B 5/7207; A61B 5/7242; A61B 5/7246; A61B 5/725; A61B 5/7267; A61B 5/7271; A61B 5/742; A61B 5/746; A61B 2503/12; A61B 2560/045

(57) ABSTRACT

An apparatus for performing an activity based on a sympathetic nervous system (SNS) response to a stimulus includes an electrode configured to contact a person undergoing the stimulus. The apparatus also includes a processing unit having an algorithm programmed by machine-learning and configured to: (i) receive at least one of electrocardiogram (ECG) signals or electrodermal activity (EDA) signals from the electrode; (ii) analyze at least one of skin sympathetic nerve activity (SKNA) signals derived from the ECG signals or the EDA signals to measure the SNS response and provide an SNS response measurement; and (iii) transmit an SNS response measurement signal conveying the SNS response measurement to initiate the activity. The apparatus also includes an activity device configured to perform the activity in response to the SNS response measurement signal.

### CROSS-REFERENCE TO RELATED APPLICATIONS

This application claims the benefit of U.S. Provisional Patent Application No. 63/500,810, filed on May 8, 2023, the contents of which is incorporated herein by reference in its entirety.

### STATEMENT OF GOVERNMENT SUPPORT

This invention was made with government support under grant number N00014-21-1-2255 awarded by the Office of Naval Research (ONR). The government has certain rights in the invention.

### TECHNICAL FIELD

Methods and apparatus for evaluation of the sympathetic nervous system (SNS) of a subject using skin sympathetic nerve activity (SKNA) derived from electrocardiogram (ECG) signals and applications thereof.

### BACKGROUND

The sympathetic nervous system (SNS) is one of the branches of the autonomic nervous system (ANS), which is invoked during situations demanding heightened energy and responses to stress or emergencies. Activation of the sympathetic nervous system leads to dilation of pupils, constriction of blood vessels, heightened sweat secretion, and suppression of digestive contractions. The significance of SNS in various conditions such as hypertension, coronary artery disease, and heart failure is firmly established and supported by extensive documentation. Objective assessment of SNS activity can provide insight into these conditions and facilitate the development of proper interventions and treatments.

Traditionally, SNS has been assessed using different approaches, including microneurography, heart rate variability (HRV) derived from electrocardiogram (ECG), and electrodermal activity (EDA). Microneurography, established as the gold standard for evaluating sympathetic nerve activity (SNA), can directly record the electrical activity of individual nerve fibers. However, due to its invasive nature and delicate skills needed for inserting the electrode needles, alternative indirect and non-invasive methods such as heart rate variability (HRV) and especially electrodermal activity (EDA) have gained popularity as it has shown higher sensitivity in assessing the SNS. EDA signal reflects the changes in skin conductance, primarily influenced by the activity of sweat glands that are solely innervated by the SNS, rendering EDA particularly sensitive in capturing sympathetic arousal dynamics, even with relatively shorter data lengths compared to HRV.

HRV, derived from the ECG signal, has shown its effectiveness when assessing sympathovagal balance at the sinoatrial level. However, HRV is unable to provide detailed second-by-second temporal resolution and it is well documented that SNS cannot be accurately assessed in the low frequency band, which also reflects parasympathetic nervous activity. Moreover, it is problematic to use HRV for patients with irregular heartbeats, such as arrhythmia. There have been several alternative approaches to separate sympathetic and parasympathetic nervous systems from HRV but they require further validation.

Accordingly, there remains a need in the art for new and effective methods to accurately evaluate the sympathetic nervous system through non-invasive means.

### SUMMARY

Disclosed is an apparatus for performing an activity based on a sympathetic nervous system (SNS) response to a stimulus. The apparatus includes: an electrode configured to contact a person undergoing the stimulus; a processing unit having an algorithm programmed by machine-learning and configured to: (i) receive at least one of electrocardiogram (ECG) signals or electrodermal activity (EDA) signals from the electrode; (ii) analyze at least one of skin sympathetic nerve activity (SKNA) signals derived from the ECG signals or the EDA signals to measure the SNS response and provide an SNS response measurement; and (iii) transmit an SNS response measurement signal to initiate the activity in response to the SNS response measurement; and an activity device configured to perform the activity in response to the SNS response measurement signal.

Also disclosed is a non-transitory computer-readable medium storing instructions that, when executed by a processor, cause the processor to perform a method for measuring a sympathetic nervous system (SNS) response to a stimulus. The method includes: receiving at least one of electrocardiogram (ECG) signals or electrodermal activity (EDA) signals from the electrode; analyzing at least one of skin sympathetic nerve activity (SKNA) signals derived from the ECG signals or the EDA signals to measure the SNS response and provide an SNS response measurement; and transmitting an SNS response measurement signal to initiate the activity in response to the SNS response measurement.

Further disclosed is a method for performing an activity based on a sympathetic nervous system (SNS) response to a stimulus. The method includes: receiving at least one of electrocardiogram (ECG) signals or electrodermal activity (EDA) signals from an electrode; analyzing at least one of skin sympathetic nerve activity (SKNA) signals derived from the ECG signals or the EDA signals to measure the SNS response and provide an SNS response measurement; transmitting an SNS response measurement signal to initiate the activity in response to the SNS response measurement; and performing the activity using an activity device in response to the SNS response measurement signal.

In one aspect, disclosed herein is a system for assessing sympathetic nervous system (SNS) activity, including: an ECG electrode for recording ECG signals from a subject and a processing unit configured to receive baseline ECG signals while the subject is not experiencing an external stimulus, receive stimulated ECG signals while the subject is experiencing an external stimulus, process the ECG signals to extract SKNA, calculate iSKNA from the extracted SKNA, extract features from the iSKNA signals, and compare the extracted features corresponding to the baseline ECG signals to the stimulated ECG signals to assess the SNS activity.

In some embodiments, the system further includes a display for visualizing the extracted features and the assessment of SNS activity.

In some embodiments, the processing unit is further configured to: segment the iSKNA signals into time windows corresponding to baseline and stimulation periods and compare the extracted features between the baseline and stimulation segments to assess the SNS activity.

In some embodiments, the processing unit is further configured to: train a machine learning model using the extracted features from the iSKNA signals, and use the trained machine learning model to classify the SNS activity in real-time based on new iSKNA signals.

In some embodiments, the processing unit is further configured to: apply a motion artifact reduction algorithm to the ECG signals before extracting the SKNA to reduce the impact of subject movement on the SNS activity assessment.

In some embodiments, the system further includes a remote device, and wherein the processing unit is further configured to: generate a report summarizing the SNS activity assessment; and transmit the report to the remote device for review by a healthcare provider.

In another aspect, disclosed herein is a non-transitory computer-readable medium storing instructions that, when executed by a processor, cause the processor to perform a method for assessing sympathetic nervous system (SNS) activity, the method includes, receiving baseline electrocardiogram (ECG) signals recorded from a subject using an electrode placed on the subject's body, receiving stimulated electrocardiogram (ECG) signals recorded from the subject using the electrode placed on the subject's body, processing the ECG signals to extract skin sympathetic nerve activity (SKNA) by applying a bandpass filter to the ECG signals, calculating an integral of the SKNA (ISKNA) by rectifying the filtered SKNA signals and applying a moving average filter, extracting features from the iSKNA signals, and comparing the extracted features corresponding to the baseline ECG signals to the stimulated ECG signals to assess the SNS activity.

In yet another aspect, disclosed herein is a method for assessing sympathetic nervous system (SNS) activity, including: recording baseline electrocardiogram (ECG) signals from a subject using an electrode placed on the body of the subject, recording stimulated electrocardiogram (ECG) signals from the subject using the electrode placed on the body of the subject, processing the ECG signals to extract skin sympathetic nerve activity (SKNA) by applying a bandpass filter to the ECG signals, calculating an integral of the SKNA (ISKNA) by rectifying the filtered SKNA signals and applying a moving average filter, extracting features from the iSKNA signals; and comparing the extracted features corresponding to the baseline ECG signals to the stimulated ECG signals to assess the SNS activity.

In some embodiments, the ECG signals are recorded at a sampling frequency of 10 kHz using two channels.

In some embodiments, the electrode is placed on the subject's wrist, chest, or ribs.

In some embodiments, the bandpass filter applied to the ECG signals has a range of 500-1000 Hz or 1700-2000 Hz.

In some embodiments, the moving average filter applied to the rectified SKNA signals has a time window between 50 ms and 100 ms with an overlap between 25 ms and 75 ms.

In some embodiments, the extracted features are selected from the group having: peak amplitude, average amplitude, standard deviation, and cumulative duration of high-amplitude intervals.

In some embodiments, the high-amplitude intervals are determined using the average amplitude of the entire iSKNA signal.

In some embodiments, the method further includes segmenting the iSKNA signals into time windows.

In some embodiments, the time windows for segmentation are between 5 s and 100 s.

In some embodiments, the method of claim 4, further includes training a machine learning model using the extracted features, and using the trained model to classify the SNS activity in real-time.

In some embodiments, the machine learning model is selected from the group having: K-nearest neighbors (KNN), support vector machine (SVM), decision tree (DT), random forest (RF), and multi-layer perceptron (MLP).

In some embodiments, the method further includes optimizing the hyperparameters of the machine learning model using grid search or random search.

In some embodiments, the method further includes evaluating the discriminative power of the extracted features using Fisher's ratio and the area under the receiver operating characteristic curve (AUROC).

### BRIEF DESCRIPTION OF THE DRAWINGS

The features, aspects, and advantages of the present technology will become better understood with regards to the following figures:

FIG. 1 provides steps to an exemplary method in accordance with the present disclosure;

FIG. 2 provides a diagram of an exemplary system in accordance with the present disclosure;

FIG. 3 provides a representative smoothed iSKNA signal (window=1 s) and its corresponding EDAphasic during the VM test, b1 to b4 mark the start of a stimulus;

FIG. 4 provides a representative iSKNA signal and its corresponding EDAphasic during the Stroop test;

FIG. 5 provides a representative iSKNA signal and its corresponding EDAphasic during the pain test. The vertical dotted lines mark the intervals during which the subject's hand was in contact with the grills and pain was present;

FIG. 6 provides an EDA time-series and their corresponding smoothed iSKNA signal (window=3 s). b1 to b4 mark the start of a stimulus;

FIG. 7 provides zero-centered iSKNA signals with two different window sizes and the corresponding synchronized EDAphasic signal. The dotted line indicates the original onset of the EDAphasic signal;

FIG. 8 is a table providing a summary of Correlation Coefficients between EDAphasic and iSKNA Before and After Synchronization and Smoothing (win=4 s) Captured during the VM Experiment;

FIG. 9 is a table providing Delay of EDAphasic Signal Relative to iSKNA Signal Captured during the VM Experiment;

FIG. 10 provides a comparative box plots of SKNA indices (channel 1) before and during the VM bursts;

FIG. 11 is a table providing SKNA and EDA Indices and Statistical Tests Results from VM Test;

FIG. 12 provides comparative box plots of EDA indices (MTVSymp) before and during the VM bursts;

FIG. 13 is a table providing SKNA and EDA Indices and Statistical Tests Results from Stroop Test;

FIG. 14 provides comparative box plots of SKNA indices (channel 1) before and during the Stroop test;

FIG. 15 provides comparative box plots of EDA indices (EDAphasic) before and during the Stroop test;

FIG. 16 is a table providing SKNA Indices and Statistical Tests Results from Pain Test;

FIG. 17 is a table providing EDA Indices and Statistical Tests Results from Pain Test;

FIG. 18 provides comparative box plots of SKNA indices (channel 1) before and during the pain inductions;

FIG. 19 provides comparative box plots of EDA indices (EDAphasic) before and during the pain inductions;

FIG. 20 is a table providing correlations between Different Features and Subjects' Pain Ratings (VAS);

FIG. 21 provides correlations between S.D of iSKNA (channel 1) and subjects' pain ratings (VAS);

FIG. 22 is a table providing classification Summary for The VM Test;

FIG. 23 provides confusion matrices for the VM test;

FIG. 24 is a table providing classification summary for the Stroop test;

FIG. 25 provides confusion matrices for the Stroop test;

FIG. 26 is a table providing classification summary for the pain test;

FIG. 27 provides confusion matrices for the pain test;

FIG. 28 provides an iSKNA signal and its corresponding EDAphasic during the pain test improper EDA response;

FIG. 29 provides representative examples of TVSymp for CNS-OT and non-CNS-OT exposures. The thick rectangular red line represents motion artifact detection using the referenced algorithm;

FIG. 30 provides a histogram of TVSymp peaks in the first and last five minutes of EDA recordings;

FIG. 31 is a table providing machine learning results on seizure detection;

FIG. 32 provides TVSymp characteristics for “Definite” CNS-OT exposure and ≥50% “Probable” exposure;

FIG. 33 provides prediction time using machine learning on human subject data (the redline shows the median value);

FIG. 34 depicts aspects of a pain alleviation system;

FIG. 35 depicts aspects of an oxygen-toxicity detection system; and

FIG. 36 is a flowchart for a method for performing an activity based on a sympathetic nervous system (SNS) response to a stimulus.

### DETAILED DESCRIPTION

The technology disclosed herein relates to methods and apparatus for assessing sympathetic nervous system (SNS) activity by measuring electrodermal activity (EDA) and skin sympathetic nerve activity (SKNA).

SKNA can be extracted from high-frequency electrocardiogram (ECG) recordings using a technique called NeuECG. The technology disclosed herein provides methods and apparatus for effectively using EDA and SKNA in assessing SNS activity under various sympathetic stimulations, including stimulations involving cognitive stress and pain. In some embodiments, EDA and SKNA signals are simultaneously recorded from individuals undergoing these stimulations, relevant features are extracted from the signals, and statistical analysis is performed. Classification techniques developed using machine learning are used to evaluate the effect of stimuli on the SKNA.

The present disclosure provides for assessing SNS activity by using EDA and SKNA data thereby providing improved diagnosis, monitoring, and treatment of conditions associated with sympathetic nervous system dysfunction. Aspects including the data acquisition process, signal processing techniques, feature extraction methods, and comparative analysis procedures are introduced below.

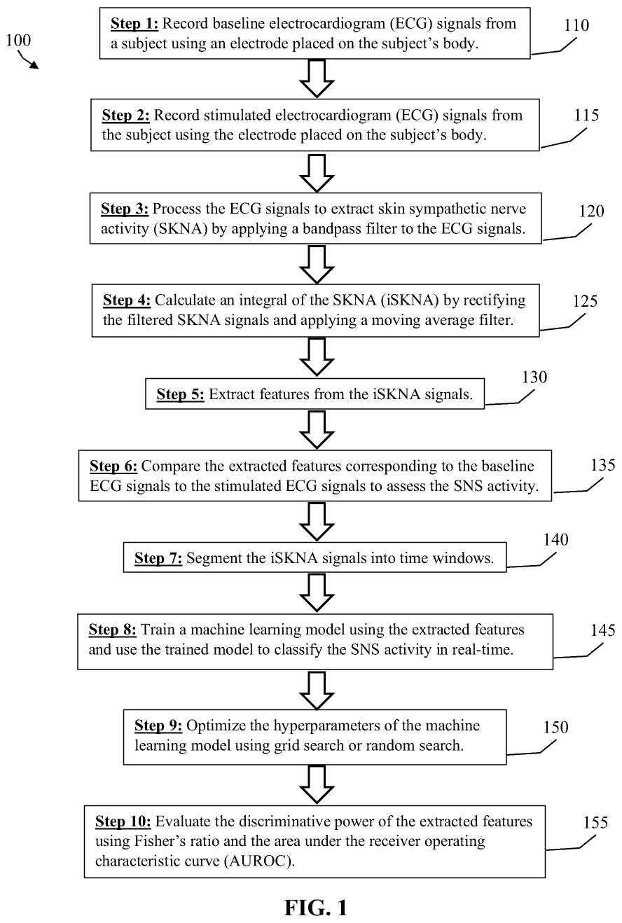

An exemplary method (100) is provided in FIG. 1. The exemplary method (100) commences with: recording baseline electrocardiogram (ECG) signals from a subject using an electrode placed on the subject's body (110); subsequently, recording stimulated electrocardiogram (ECG) signals from the subject using the electrode placed on the body (115) of the subject; processing the ECG signals to extract skin sympathetic nerve activity (SKNA) by applying a bandpass filter to the ECG signals (120); calculating an integral of the SKNA (ISKNA) by rectifying the filtered SKNA signals and applying a moving average filter (125); extracting features from the iSKNA signals (130); and comparing the extracted features corresponding to the baseline ECG signals to the stimulated ECG signals to assess the SNS activity (135); segmenting the iSKNA signals into time windows (140); training a machine learning model using the extracted features and using the trained model to classify the SNS activity in real-time (145); optimizing the hyperparameters of the machine learning model using grid search or random search (150); and evaluating the discriminative power of the extracted features using Fisher's ratio and the area under the receiver operating characteristic curve (AUROC) (155).

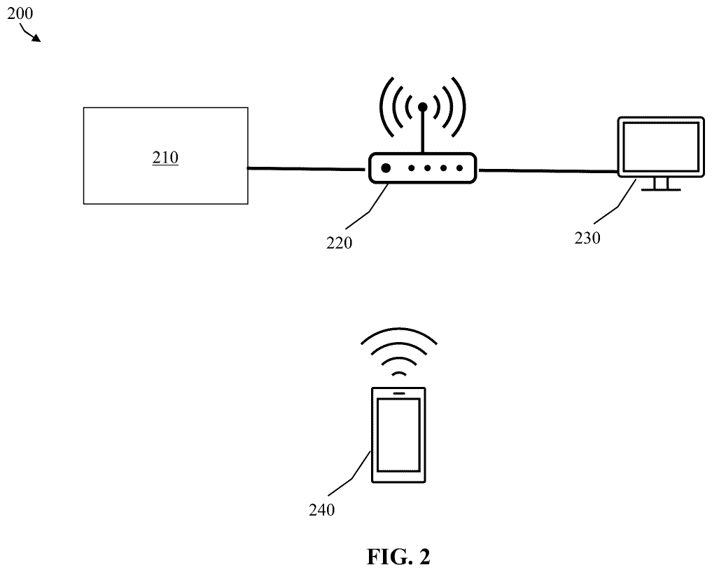

An exemplary system (200) is provided in FIG. 2. The exemplary system (200) includes an ECG electrode (210) for recording ECG signals from a subject, a processing unit (220), a display (230) for visualizing the extracted features and the assessment of SNS activity, and a remote device (240) to receive a report from the processing unit for review by a healthcare provider. The processing unit (220) may also be referred to as a “processor” and by other similar terms.

The processing unit (220) may be configured with non-transitory machine-readable media for executing various instructions. For example, the processing unit (220) may be configured to receive baseline ECG signals while the subject is not experiencing an external stimulus, receive stimulated ECG signals while the subject is experiencing an external stimulus, process the ECG signals to extract SKNA, calculate iSKNA from the extracted SKNA, extract features from the iSKNA signals, and compare the extracted features corresponding to the baseline ECG signals to the stimulated ECG signals to assess the SNS activity.

### 1. Definitions

As used herein, the term “sympathetic nervous system” or “SNS” generally refers to portions of the autonomic nervous system responsible for regulating involuntary physiological processes, particularly those associated with the “fight or flight” response of a body to stress, danger, or increased arousal. The sympathetic nervous system may innervate various organs and glands throughout the body, including the heart, blood vessels, lungs, pupils, sweat glands, and digestive system. When activated, the sympathetic nervous system may trigger a series of physiological changes, such as increased heart rate, blood pressure, and respiration, dilation of pupils, increased sweating, and decreased digestive activity.

As used herein, the term “electrocardiogram” or “ECG” generally refers to diagnostic tool that records the electrical activity of the heart over time. An ECG is a graphical representation of the propagation of electrical impulses through the heart muscle during each cardiac cycle as a waveform, obtained by placing electrodes on the skin surface or within the body. The ECG waveform includes several distinct components, each representing a specific phase of the cardiac cycle, including the P wave, which represents atrial depolarization; the QRS complex, which represents ventricular depolarization; and the T wave, which represents ventricular repolarization. An ECG may provide information about the rhythm, rate, and electrical conduction patterns of a heart, among others.

As used herein, the term “electrodermal activity” or “EDA” generally refers to the changes in the electrical properties of the skin, primarily due to the activity of sweat glands, which are controlled by the sympathetic nervous system. Measurement of electrical properties of the surface of the skin related to EDA generally provides a non-invasive method for evaluating response of the autonomic nervous system to various stimuli. For example, EDA may be measured by measuring skin conductance. Skin conductance may be measured by applying a small electrical current between two electrodes placed on the skin surface. Generally, EDA signals include two main components: a tonic component, which represents the slow, gradual changes in skin conductance over time, and a phasic component, which represents the rapid, transient changes in skin conductance, often in response to specific stimuli. The phasic component is further characterized by skin conductance responses (SCRs), which are discrete, short-duration increases in skin conductance.

As used herein, the term “skin sympathetic nerve activity” or “SKNA” generally refers to the electrical activity of the sympathetic nerve fibers innervating the skin. Measuring SKNA electrical signals extracted from ECG signals is a method for directly assessing the sympathetic nervous system's activity, and may be characterized by frequency, amplitude, and duration of electrical impulses of the SKNA electrical signals.

As used herein, the term “machine learning” generally refers to a class of artificial intelligence (AI) that focuses on the development of algorithms and statistical models that enable computer systems to learn and improve their performance on a specific task without being explicitly programmed. Machine learning algorithms and methods may include building a mathematical model based on sample data, known as training data, in order to make predictions or decisions. Machine learning may include such techniques such as supervised learning, where the algorithm learns from labeled training data to predict outcomes for new, unseen data; unsupervised learning, where the algorithm discovers hidden patterns or structures in unlabeled data; and reinforcement learning, where the algorithm learns through interaction with an environment, receiving rewards or penalties for its actions. Machine learning techniques may also include supporting subsystems such as decision trees, random forests, support vector machines, neural networks, and deep learning architectures, and may be applied to various tasks used to carry out the disclosed method, including classification, regression, clustering, and anomaly detection, among others. Other artificial intelligence (AI) techniques may be used.

As used herein, the term “physiological markers” generally refers to measurable biological parameters that provide information about the functioning of an individual's body systems and organs. Physiological markers can be obtained through various methods, including invasive techniques, such as blood tests and biopsies, and non-invasive techniques, such as electrocardiogramlectroencephalography, and medical imaging. Examples of physiological markers include heart rate, blood pressure, respiratory rate, body temperature, blood glucose levels, and concentrations of specific hormones, proteins, or other biochemical compounds in bodily fluids. Physiological markers can also encompass electrical signals generated by the body, such as those measured by electrocardiogram (ECG) or electrodermal activity (EDA), which provide information about the functioning of the cardiovascular and autonomic nervous systems, respectively. In the context of the present disclosure, relevant physiological markers may include skin sympathetic nerve activity (SKNA), heart rate variability (HRV), and EDA indices, such as skin conductance level and skin conductance responses.

As used herein, the term “pain” refers to an unpleasant sensory and emotional experience associated with actual or potential tissue damage, or described in terms of such damage. The phenomenon can be characterized by its intensity, duration, location, and quality, and may be classified as acute, which is typically short-lived and results from injury, surgery, or illness, or chronic, which persists for an extended period and may be associated with ongoing pathological processes or central sensitization. Pain perception involves the activation of specialized sensory neurons called nociceptors, which detect potentially harmful stimuli and transmit signals to the central nervous system. In the context of the present disclosure, pain has been shown to elicit a robust sympathetic response, which can be measured through techniques such as skin sympathetic nerve activity (SKNA) and electrodermal activity (EDA).

### 2. Recording ECG and EDA Signals

In some embodiments, EDA signals are recorded from the index and middle fingers of the right hand of a subject using appropriate electrodes. In some embodiments, two channels of ECG signals are recorded at a sampling frequency of 10 kHz. For the first ECG channel (channel 1), electrodes may be placed on both inner wrists (Lead I). For the second channel (channel 2), the positive and negative electrodes may be positioned on the top left side of the chest and below the right rib, respectively (Lead III). The ground electrode for both channels may be attached under the left rib.

In some embodiments, EDA and ECG data is recorded using GSR Amp and Bio Amp, respectively, with PowerLab and LabChart Pro 7, and subsequently may be processed using MATLAB R2022. The sensitivity of different PowerLab ECG channels may vary, thus, the two channels that demonstrated high signal quality may be chosen heuristically. Generally, POWERLAB refers to a digital data acquisition device engineered to record precise, reliable, consistent data, and is available from ADInstruments, Inc. of Colorado Springs, CO. POWERLAB may connect with analog instruments using a digital to analog converter. MATLAB is a programming and numeric computing platform and is available from Math Works® of Natick, MA.

### 3. Time-Series Feature Calculation

In some embodiments, the ECG signals and thus the SKNA signals are recorded at a 10 KHz frequency to capture nerve activities that can extend beyond 2,000 Hz. Subsequently, a bandpass filter may be employed in the range of 500-1000 Hz to capture nerve activity and eliminate high-frequency noise. In other embodiments, a bandpass filter with a range of 1700-2000 Hz may be used.

In some embodiments, filtered SKNA signals may be further processed by rectifying them, and subsequently calculating iSKNA, which represents the integral area of the rectified SKNA. In some embodiments, this is achieved by applying a moving average filter with symmetric time-windows of 100 ms, wherein 50 ms were allocated to overlap in both the start and end of each window.

In some embodiments, the EDA signals may be resampled to 4 Hz, and then decomposed into phasic and tonic components using a cvxEDA method. The tonic component represents the gradual baseline activity of skin conductance, whereas the phasic component corresponds to rapid fluctuations associated with startle-like stimuli.

In other embodiments, alternative EDA decomposition techniques may be used, e.g. time-varying sympathetic (TVSymp) and modified TVSymp (MTVSymp). Signals may be sampled at 2 Hz when using these alternative techniques. The TVSymp computation may include a variable frequency complex demodulation (VFCDM) technique to reconstruct EDA signals in the frequency range of 0.08-0.24 Hz followed by a Hilbert transform to obtain instantaneous amplitudes of the reconstructed signals. MTVSymp emphasizes EDA changes caused purely by stimuli by removing baseline EDA responses from the prior segments. MTVSymp may be computed as shown equation 1.

$${MTVSymp}_{t} = ( 0 , TVSymp - {μ}_{{TVSymp}_{( t - 10 : t - 1 )}} ) \qquad ( 1 )$$

Where μ_{Tvsymp}_{_{(t-10:t-1)}} represents for average of the recent 5 s of TVSymp.

### 4. Segmentation and Feature Extraction

In some embodiments, different segment sizes may be used to account for the characteristics of the induced stimuli. For example, segment sizes of 10 s, 25 s, and 100 s. From each segment of the calculated time-series (iSKNA, EDAphasic, TVSymp, and MTVSymp), four distinct features may be extracted: peak amplitude, average amplitude, standard deviation, and the cumulative duration where the signal had higher amplitudes than a predefined threshold. For EDA time-series, the threshold may be set to 0.05 μS. For iSKNA, the threshold may be set to the average amplitude of the entire iSKNA signal. This feature may be labeled the high amplitude intervals (HaSKNA). The average amplitude and standard deviation (variation) of iSKNA may be denoted as aSKNA and vSKNA, respectively. In some embodiments, the EDA and SKNA segments may be visually inspected and segments corrupted by motion artifacts or other noise sources removed.

### Exemplification

### Example 1: EDA and SKNA Responses of Subjects Experiencing Various Sympathetic Stimulations, Including Cognitive Stress and Pain Compared and Analyzed

### 1. Participants

Sixteen healthy volunteers, aged between 20-57 years, were selected. The group included eight female and eight male subjects. The participants underwent the Valsalva maneuver (VM), the Stroop test, and the thermal grill pain test in a random order discussed below. Participants were seated during all tests.

### 2. Experiments

For the VM, participants were instructed to forcefully exhale after a deep inspiration. A two-minute baseline and control signal were recorded while the subjects were relaxed, engaging in normal breathing. Subsequently, participants performed the VM three to four times, with a fifty-second interval between each breathing episode.

The Stroop test involved a two-minute baseline recording as a control, followed by a two-minute session of the actual test. Subjects were presented with words on a smart tablet that denoted specific colors, but the text color of the word itself often differed from the written color name and was displayed against a differently colored background (e.g., the word “blue” written in yellow ink with a purple background). Note that the ink color and the color name might or might not have matched. The participants were asked to subvocalize the word's color instead of saying it aloud to minimize any possible myopotential interference.

For the pain test, three thermal grills with varying pain levels were used: sham (no pain), low pain, and high pain. Each thermal grill was designed with interlaced copper tubes set at either a warm temperature (40-50° C.) or cool temperature (18° C.). When a hand is placed on the grill, this temperature difference creates an illusion of pain in the brain without causing any tissue injury. Before the experiments, the warm temperatures of the low and high pain thermal grills were personalized for each subject, based on the visual analog scale (VAS), scaled between 0-10. The high pain grill was set to elicit a pain rating of at least 7 out of 10 on the VAS for each subject, whereas the low pain grill was set to elicit a pain rating of 4 to 6 on the VAS. The participants were blindfolded, and three thermal grills were positioned on a wheeled table in such a way that the participants were unaware of which type of grill would come into contact with their left hand. To accurately position their hands onto the thermal grills, two verbal cue signs: ‘ready’ and ‘go’ were used. For ‘ready’, an experimenter asked participants to move their left hand slightly to the left. The other experimenter adjusted the table to locate the target thermal grill right below the left hand. Upon the ‘go’ sign, participants lowered their left hand instantly onto the grill.

After personalizing the temperatures of the grills, a two-minute period for baseline recording and hemodynamic stabilization was provided. During this period, subjects were instructed to sit in a relaxed manner and minimize any movements. A sequence of nine stimuli was then conducted, with three stimuli for each thermal grill, in a randomized order, with an interstimulus interval of approximately 40 s. After each stimulus, participants reported their pain levels on a scale from 0 to 10 (VAS).

### 3. Statistical Analysis

In the VM experiment, each stimulus consistently resulted in either one or several bursts in both EDAphasic and iSKNA signals, making VM a suitable procedure to evaluate the correlation between EDAphasic and iSKNA responses. Pearson correlation coefficients were calculated between iSKNA and EDAphasic segments for the VM experiment. As EDA signals generally have a few seconds of delay in response after a stimulus, a cross-correlation technique was used during the VM test to align the onset timepoints between EDAphasic and iSKNA. This involved calculating the cross-correlation between EDAphasic and iSKNA in a segment across all possible lags to determine the delay. EDAphasic has a shorter frequency range compared to the calculated iSKNA. To account for this difference, iSKNA was also calculated using a window size of four seconds for smoothing the signal. The lmer function in R for fitting linear mixed-effects models (LMM) to each feature across the different labels was used. LMM relates with the issue of repeated measurements within subjects by considering individual differences and is used to determine whether there are significant differences in EDA and SKNA features across different classes, including baseline and distinct levels of the stimulation phase, i.e., VM, Stroop test, and different pain levels (sham, low pain, and high pain). If significant differences in the pain test were observed, post hoc analyses were performed using the estimated marginal means (EMMs) with the Tukey adjustment method. P-value <0.05 was considered significant. Pearson correlation coefficients between EDA/SKNA features and VAS scores obtained during the pain test were also calculated.

The discriminative and classification accuracy of each feature was assessed utilizing two evaluation metrics: Fisher's ratio and the area under the receiver operating characteristic curve (AUROC).

For Fisher's ratio, it was calculated as a measure of how well each feature could discriminate between each two neighboring classes (for VM: baseline vs. burst; for Stroop test: baseline vs. Stroop; for pain test: baseline vs. sham, sham vs. low pain, and low pain vs. high pain). This ratio helps to identify features that exhibit significant differences between two certain classes, making them potentially useful for classification purposes.

To calculate the AUROC, first the features of the two classes across all subjects were normalized to a range between 0 and 1. This normalization process involved subtracting the minimum value and then dividing by the maximum value. Next, multiple thresholds for each feature were generated. Subsequently, AUROC was obtained by calculating the area under this curve. AUROC evaluates the performance of each feature in distinguishing between the two classes. A higher AUROC value indicates better discriminative ability and classification accuracy for the feature associated with a chosen threshold value.

### 4. Classification

Five machine learning models were employed for data classification for all three experiments: K-nearest neighbors (KNN), support vector machine (SVM), decision tree (DT), random forest (RF), and multi-layer perceptron (MLP). To minimize over-fitting and maximize the number of samples, the leave-one-subject-out cross-validation approach was employed. In this method, the models were iteratively trained using segments from all subjects except one, then evaluated each model's performance using the excluded subject's segments.

First, the feature values were standardized to have zero mean and unit variance. Note that the features from the testing batch were standardized using mean and standard deviation values obtained from the training batch only. Considering the experimental protocol, which usually provided three labels for each class per subject, except for the baseline label, care was taken to maintain this balance.

Three baseline labels for each subject were randomly selected, ensuring consistency across classes. Next, parameter optimization for certain hyper-parameters was conducted. For K-nearest neighbors (KNN), the value of k was fine-tuned within each leave-one-subject-out iteration. Using grid search cross validation, k was selected from a range of values spanning 1 to 10. In a similar manner, the number of trees was optimized for the random forest model from the set n=[10, 50, 100, 150, 200, 250, 300, 350, 400].

For constructing the MLP model, a straightforward design involving two layers was adopted. Determining the number of neurons in each layer was accomplished via random search. One hundred iterations were conducted, each time assigning two random values ranging from 10 to 100. In the evaluation phase, the specificity and sensitivity for each class was calculated with only the test data.

### 5. Results

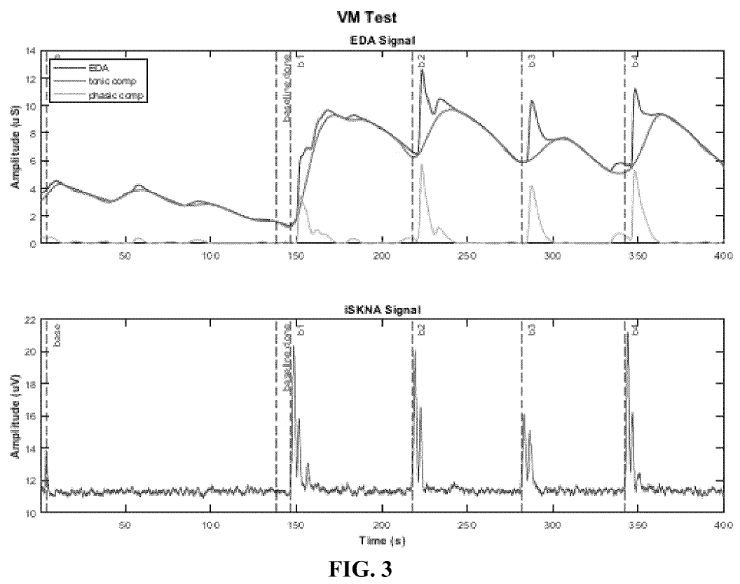

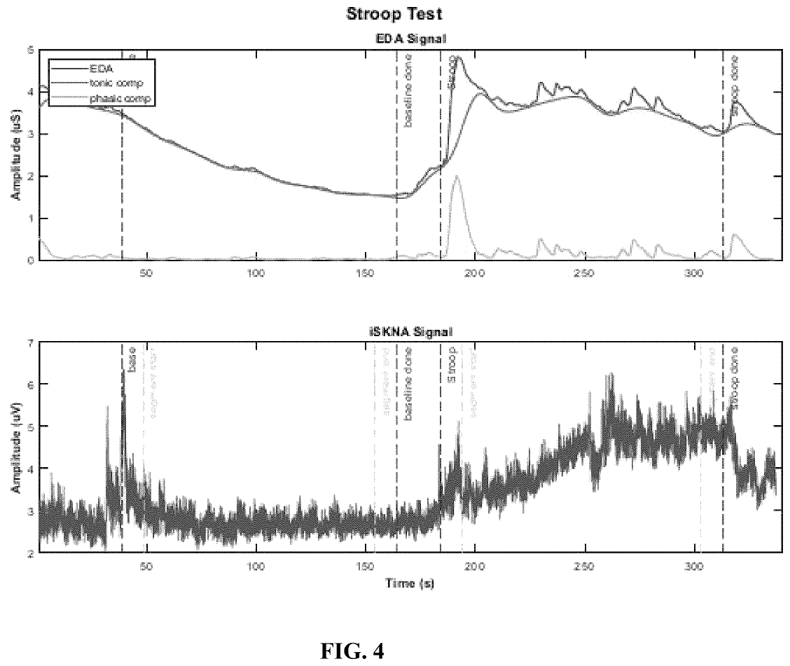

FIGS. 3 to 5 display representative EDA, including both phasic and tonic components from cvxEDA decomposition, and iSKNA signals recorded during VM, Stroop, and pain tests, respectively. iSKNA signals are observed to noticeably increase during these tests, which are designed to invoke SNS activity as previous studies have shown. Note that the iSKNA responses show an immediate reaction to stimuli, whereas EDA responses exhibit delays in their onset. Additionally, iSKNA signals demonstrate an abrupt ending of their response after stimuli, in contrast to EDA responses, which have a prolonged effect.

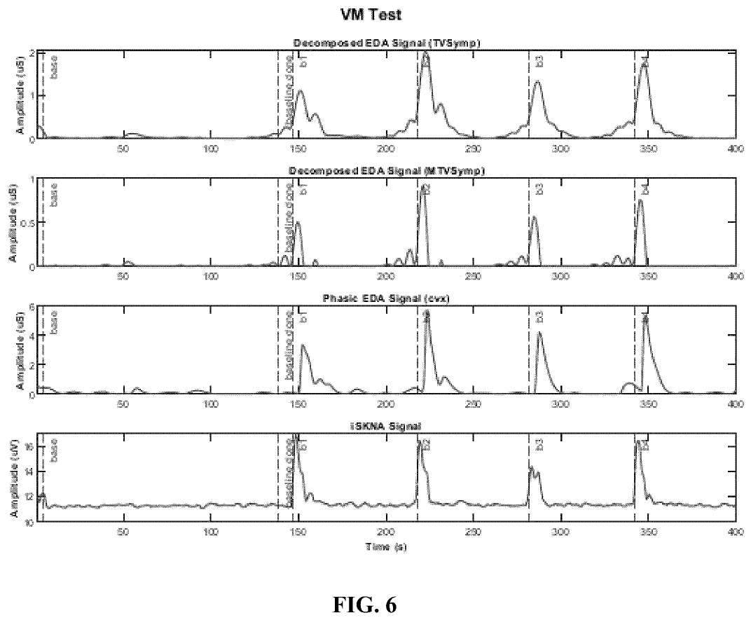

FIG. 6 depicts a representative VM illustration showing the three decompositions of EDA (TVSymp, MTVSymp, and EDAphasic) alongside their corresponding smoothed iSKNA signal, employing a window of size of three seconds.

For the Stroop test, channel 2 of the SKNA data was excluded due to relatively weak stimulations in both SKNA and EDA.

### 6. Correlation Analysis and EDA Delay

A notable delay was observed in the onset timings of EDAphasic signals compared to their corresponding iSKNA signals. Specifically, compared to iSKNA signals from channels 1 and 2, EDAphasic exhibited a delay of 4.66±2.58 s and 5.03±2.26 s, respectively.

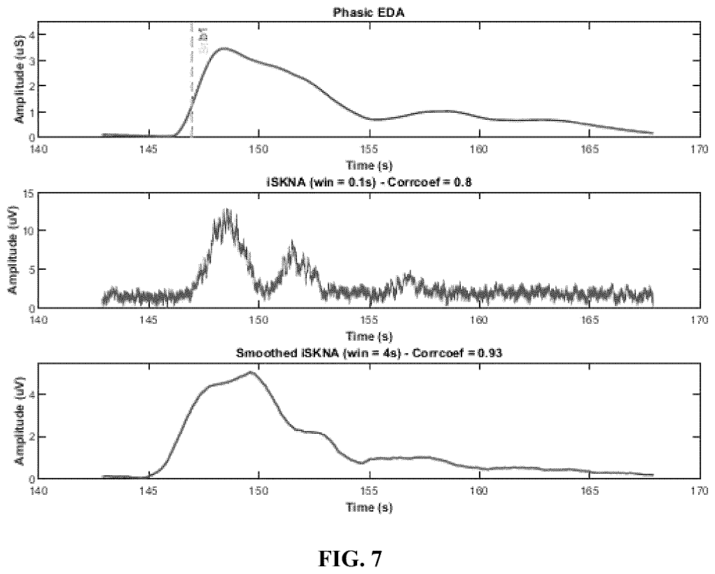

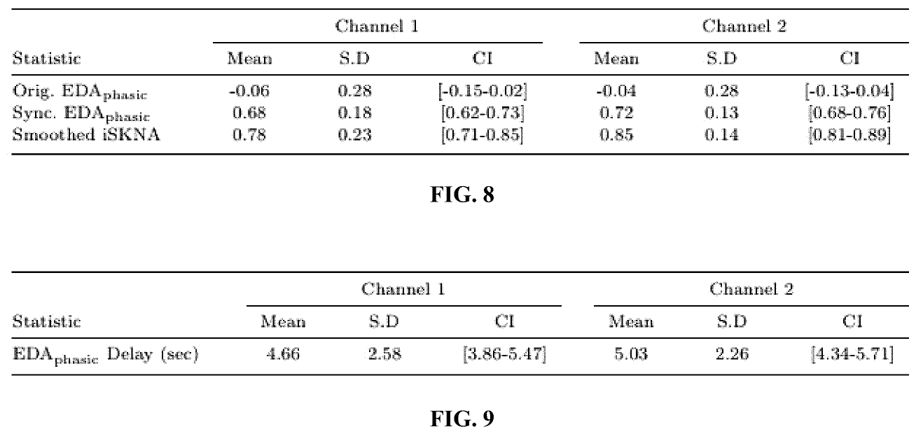

Before synchronizing the timing of onsets, a very weak correlation was found between iSKNA and EDAphasic segments, indicated by the correlation coefficients (r) of −0.06±0.28 for channel 1 iSKNA and −0.04±0.28 for channel 2 iSKNA. However, after synchronizing the onsets, the correlation coefficients significantly increased to moderate-to-high r values of 0.68±0.18 for channel 1 iSKNA and 0.72±0.13 for channel 2 iSKNA. FIG. 7 provides a representative example of EDAphasic and iSKNA bursts with synchronized response onsets. When using the four second window size for iSKNA smoothing, the correlation coefficients were further enhanced, having r values of 0.78±0.23 and 0.85±0.14 for channels 1 and 2, respectively. FIG. 8 summarizes the improvements in correlation coefficients with adjustment of the onsets and increasing the window size. FIG. 9 summarizes the delays observed in the EDAphasic signals in relation to their corresponding iSKNA signals.

### 7. Valsalva Maneuver

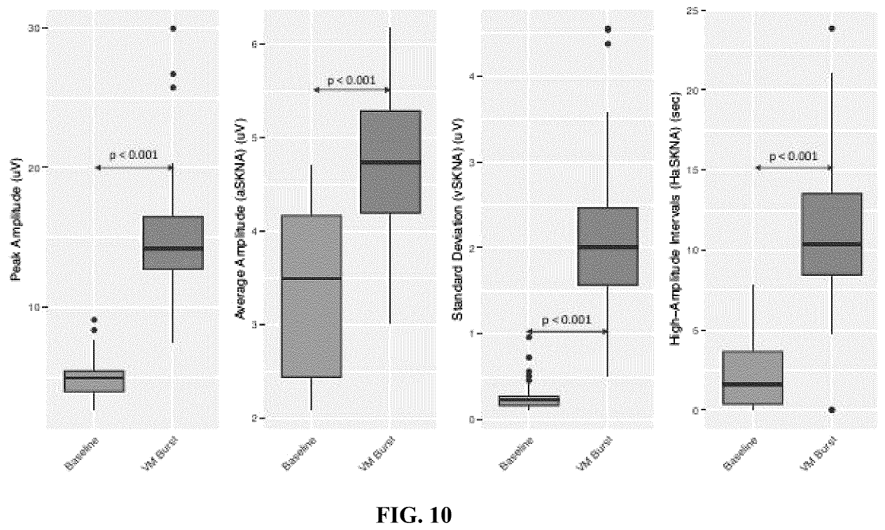

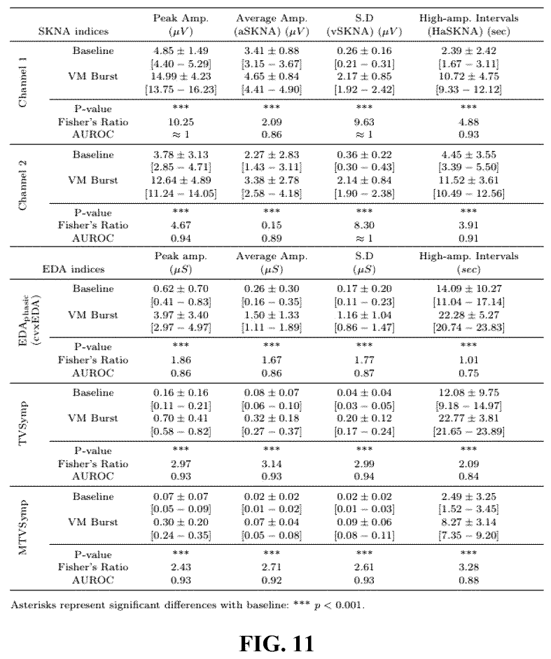

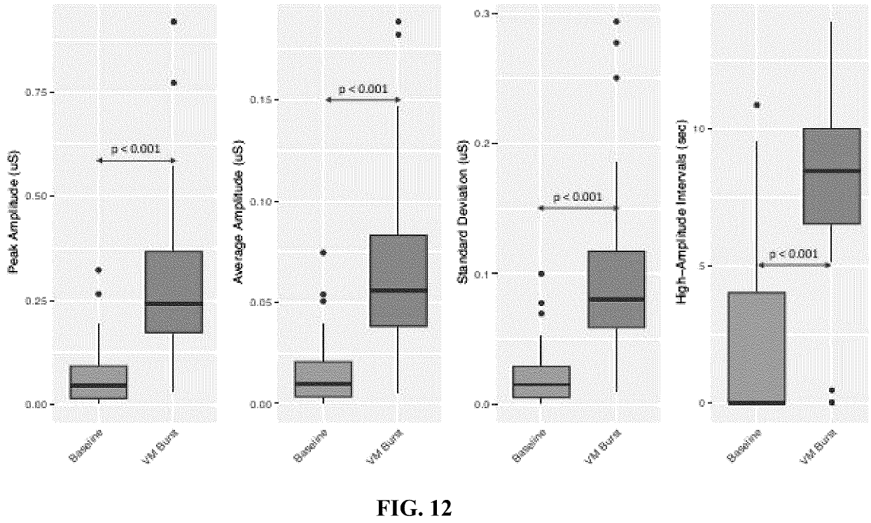

FIG. 10 shows box plots of SKNA indices derived from the iSKNA signals, which were calculated from channel 1, before and during the VM procedures. FIG. 12 illustrates the corresponding indices of EDA, extracted from the MTVSymp time-series. MTVSymp is shown since it demonstrated better results than either TVSymp or EDAphasic, as presented in FIG. 11. Inspecting the box plots, coupled with the reported p-values <0.001, reveals the robust performance of the extracted features.

FIG. 11 presents a comparison of the extracted features (peak amplitude, average amplitude, standard deviation, and the high amplitude intervals) derived from iSKNA, EDAphasic, TVSymp, and MTVSymp time-series between baseline and during the VM. Upon closer examination of each feature's discriminatory power in FIG. 11, it is evident that SKNA features, especially from channel 1, outperform the EDA features across the majority of features, except for the average amplitude feature, which has a greater Fisher's value for TVSymp. The computed Fisher's ratios consistently demonstrate that the between-class variances remain smaller than the within-class variances across all features, with the exception of aSKNA from channel 2. This consistent trend highlights the inherent potential of most features for effective classification. Among the SKNA features, the peak value stands out with an impressive Fisher's ratio of 10.25 for channel 1 and 4.67 for channel 2, closely followed by vSKNA with ratios of 9.63 for channel 1 and 8.30 for channel 2. It is noteworthy that the same set of features consistently yielded higher AUROCs across all signals, notably AUROC≈1 for both iSKNA peak value (channel 1) and vSKNA (channel 1 and 2).

### 8. Stroop Test

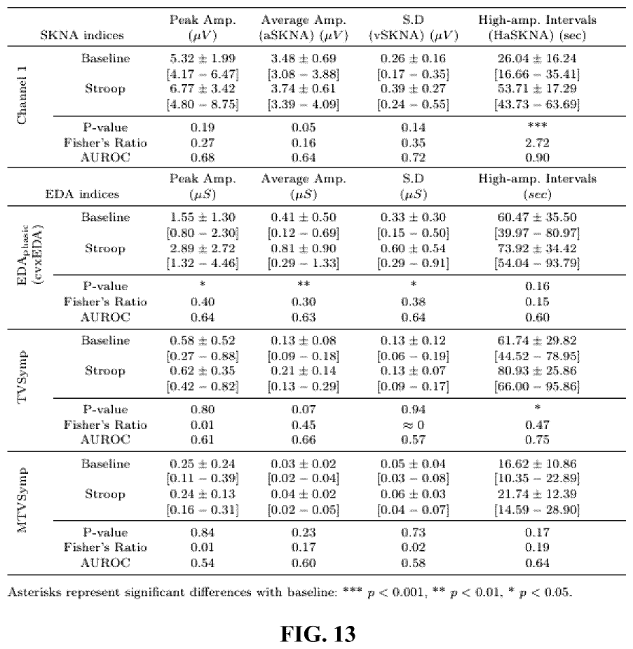

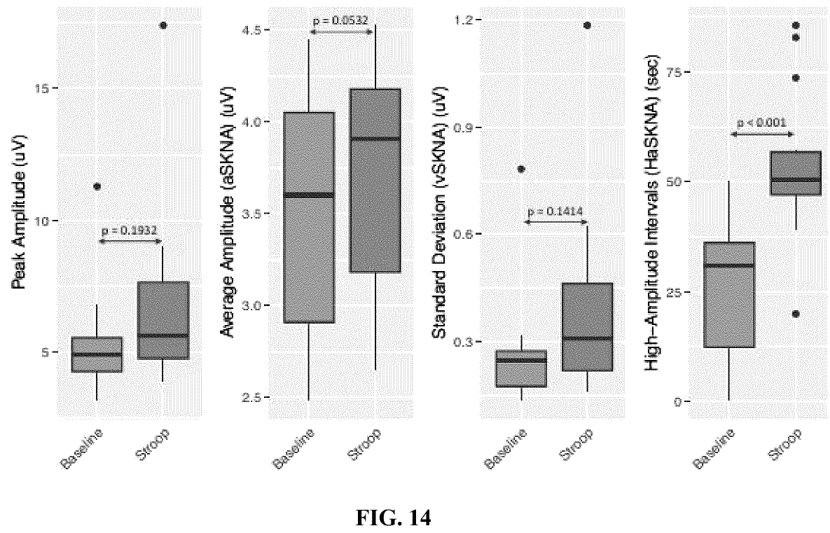

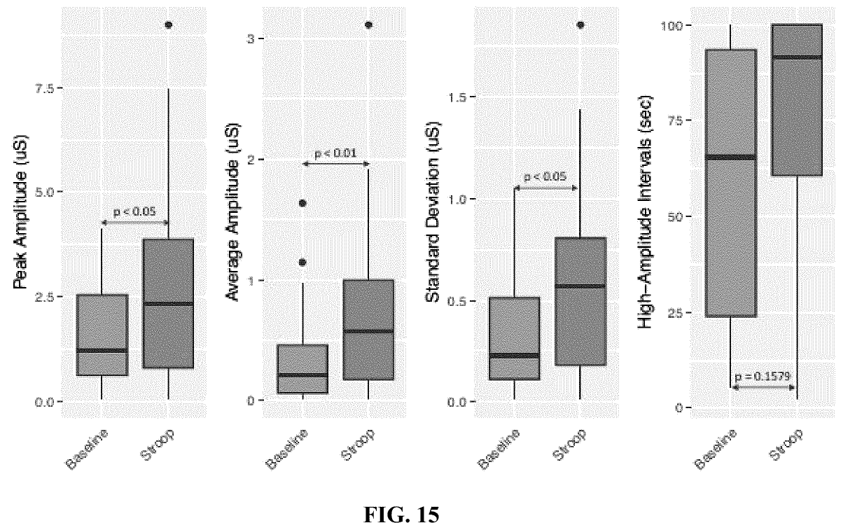

FIG. 13 displays the SKNA, EDA indices, and statistical tests results from the Stroop test. FIGS. 14 and 15 provide a comprehensive visual representation of the SKNA and EDA features during the Stroop test. P-values, Fisher's ratios and AUROC values computed for SKNA features indicate that the sole noteworthy feature exhibiting substantial discriminatory power is HaSKNA, with a value of 2.72 for Fisher's ratio and 0.90 for AUROC. Within EDA decompositions, peak amplitude, average amplitude, and standard deviation of the EDAphasic signal, and high amplitude intervals of the TVSymp signal, have p-values <0.05; however, the corresponding Fisher's ratios suggest bigger within-class variances than the between-class variances.

### 9. Thermal-Grill Pain Test

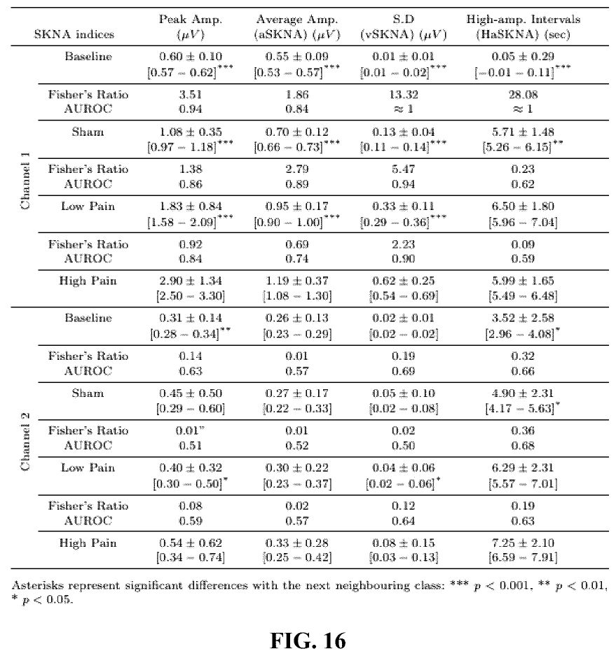

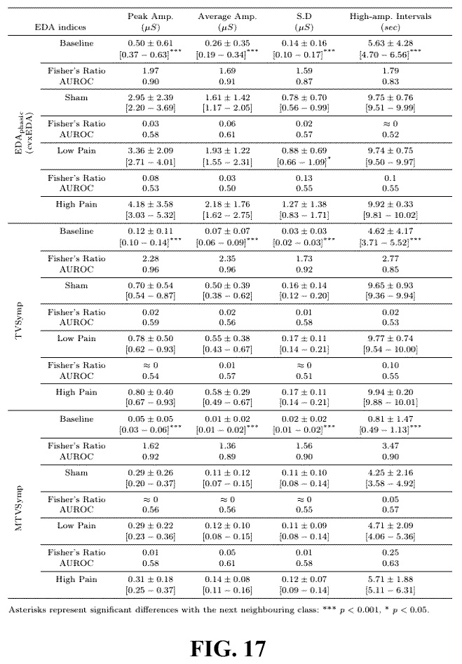

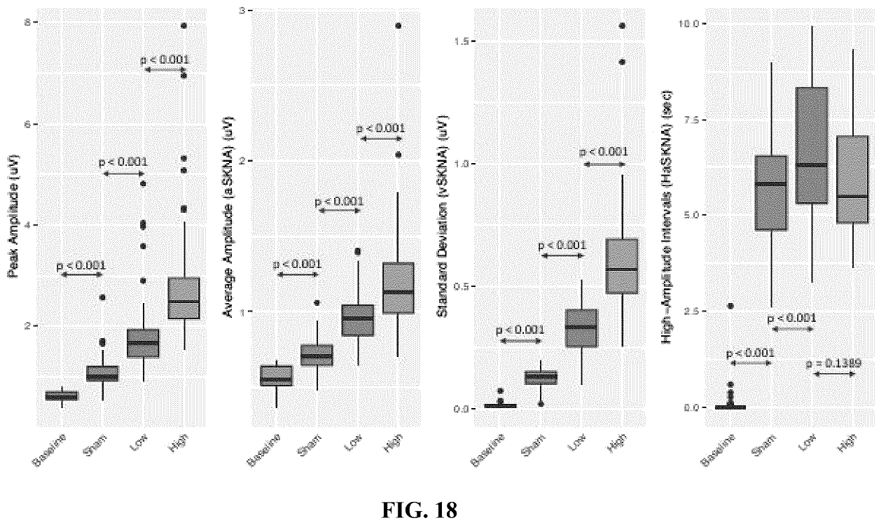

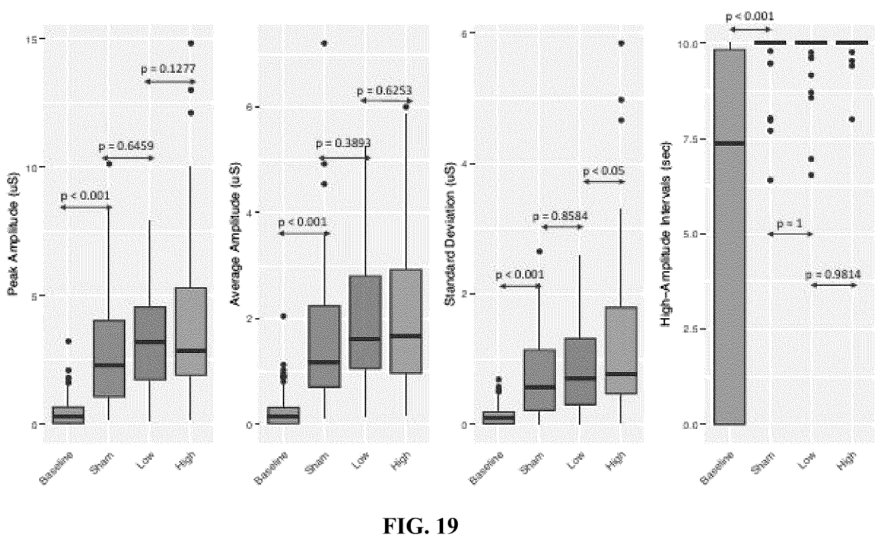

FIGS. 16 and 17 provide a comprehensive overview of the SKNA and EDA indices during the pain test, accompanied by the outcomes of the statistical analyses performed on the neighboring classes (baseline vs. sham, sham vs. low pain, and low pain vs. high pain). Further insights are visualized in FIGS. 18 and 19, presented as box plots.

Analyzing SKNA features from channel 1, it becomes evident that the peak value, aSKNA, and vSKNA exhibit notable discrepancies in mean values between all the adjacent classes. In addition, vSKNA has an AUROC of approximately 1 and Fisher's ratio=13.32 to discriminate between baseline and sham.

HaSKNA shows a significant mean difference between baseline and sham and also sham and low pain conditions. This distinction is also visually depicted in the box plot presented in FIG. 18, wherein baseline values show a marked decrease compared to segments recorded during pain induction. Conversely, no significant difference is observed between low and high pain, in this particular feature (HaSKNA). This pattern may be attributed to subjects rapidly withdrawing their hands from the higher-pain grills. Consequently, due to the brevity of stimuli in such cases, this feature demonstrates limited discriminative power within the analysis.

Considering observations from channel 2, the differentiation between baseline and sham is only statistically significant for the peak amplitude and HaSKNA, with p-values below 0.01 and 0.05, respectively. On the other hand, HaSKNA may be a good feature to distinguish between sham and low pain. However, when considering low and high pain test results, peak amplitude and vSKNA features have significant p-values, whereas the AUROC and Fisher's ratios are not considerable.

Based on the data presented in FIG. 17, it is evident that all EDA features exhibit good discriminatory ability between baseline and sham conditions, as evidenced by AUROC values exceeding 0.80, Fisher's ratios surpassing 1.36, and p-values falling below 0.001. However, when considering the discrimination of other neighboring classes, EDA features may not demonstrate strong classification performance.

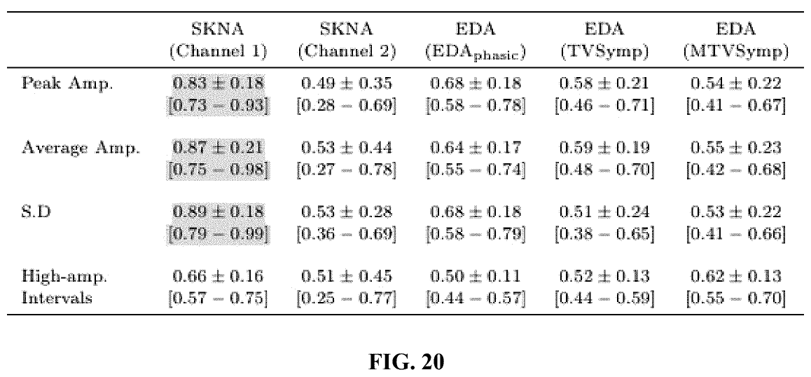

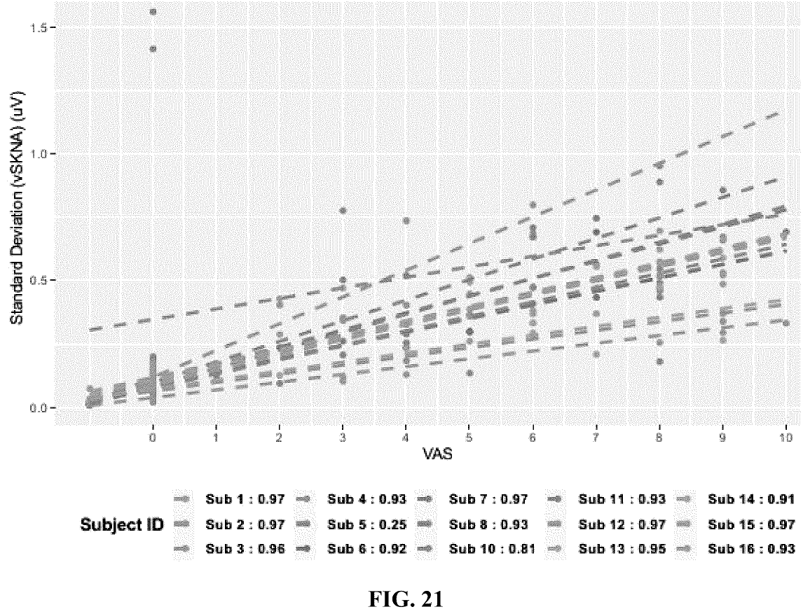

The analysis further revealed strong correlations between the pain ratings (VAS) that participants had reported during the Pain test and the extracted features. Specifically, the peak amplitude with correlation coefficient (r) of 0.83±0.18, aSKNA with r value of 0.87±0.21, and vSKNA with r value of 0.89±0.18 from channel 1 provide considerably high correlations. A comprehensive overview of these correlations is presented in FIG. 20. FIG. 21 portrays the relationship between vSKNA recorded from channel 1 and VAS pain ratings. Notably, each distinct dotted line represents the correlation trend-line for a particular subject. It's noteworthy that subject 5 exhibits two outlier samples which contribute to the lower correlation.

Conversely, apart from this isolated instance, all other subjects exhibit correlations exceeding 0.81 between the standard deviation (S.D) and VAS pain ratings.

### 10. Classification Results

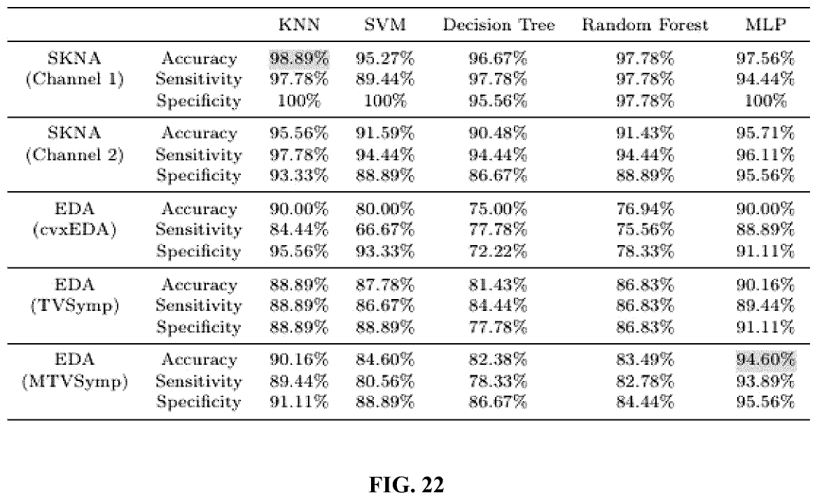

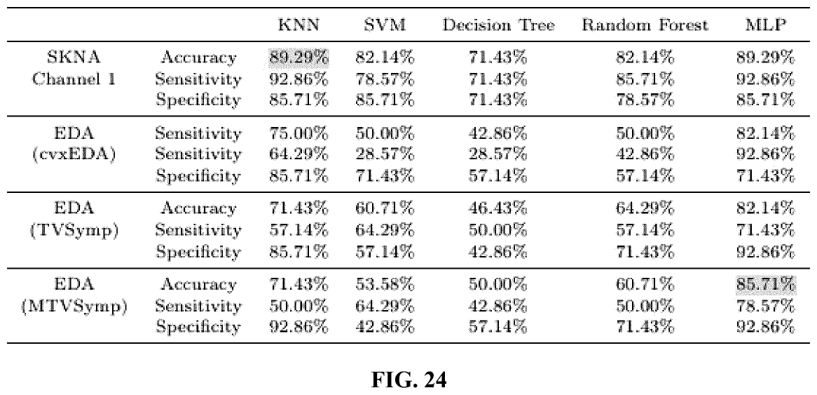

FIGS. 22, 24, and 26 provide an overview of the classification results. These tables summarize the performance based on the time-series from which features were extracted and the specific machine learning models employed.

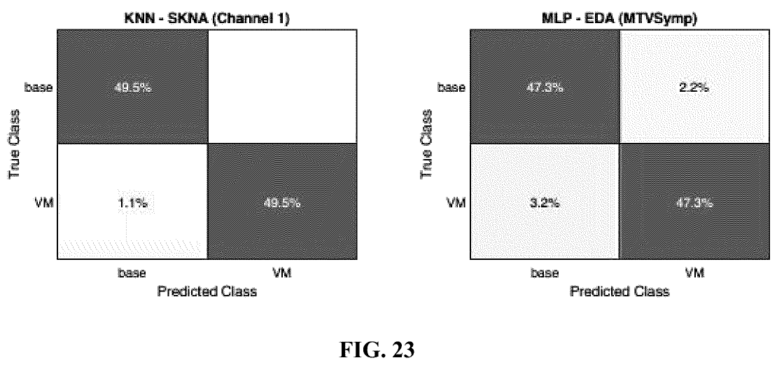

Consistently across all scenarios, SKNA features from channel 1 exhibit superior performance. Specifically, for the VM test, the optimal classification accuracy of 98.89% was achieved with the KNN model. The confusion matrices of the most proficient models for SKNA and EDA are illustrated in FIG. 23.

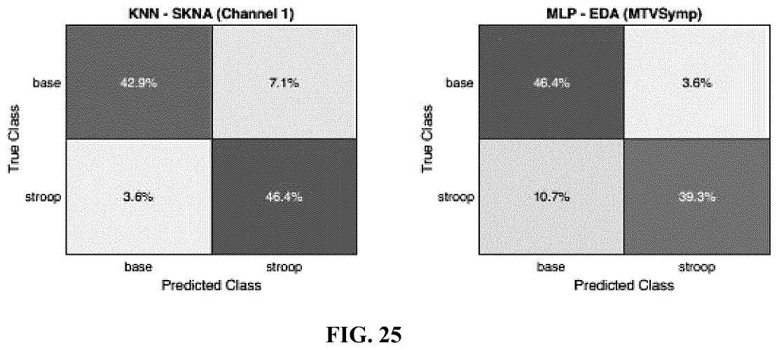

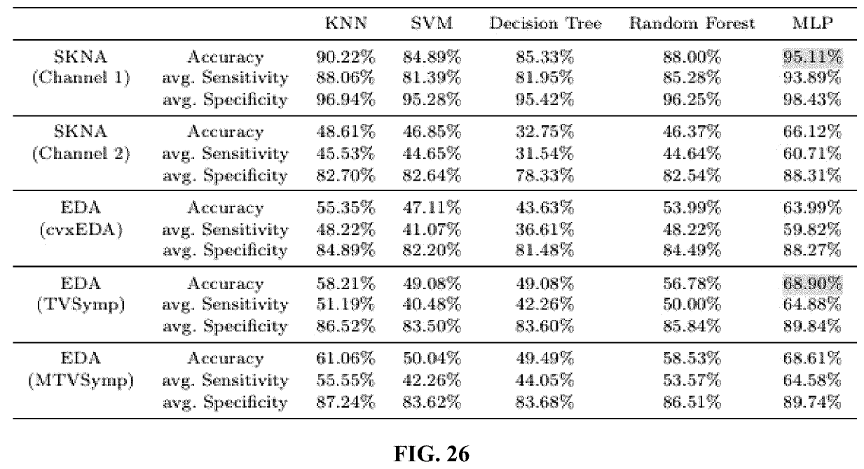

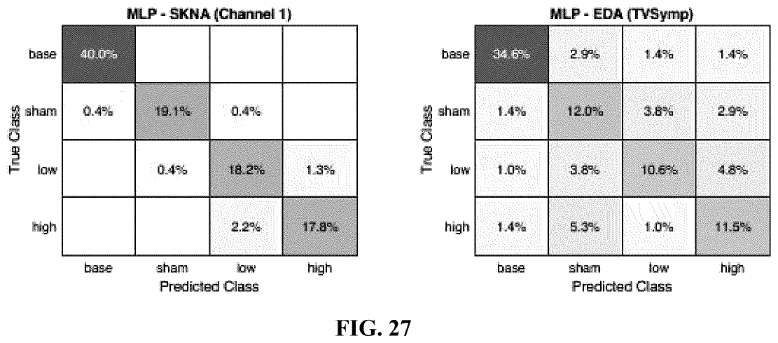

For the Stroop test, KNN provided an accuracy of 89.29% when trained using the SKNA features from channel 1. Transitioning to the pain test, the MLP model emerged as the most effective choice for the same signal, achieving an accuracy of 95.11%. FIGS. 25 and 26 visually present the confusion matrices corresponding to the optimal models trained on the features from each of these two signals.

### 11. Discussion

Both EDA and SKNA-derived features statistically increased during these tests, compared to baseline. However, SKNA outperformed EDA in terms of classification accuracy and statistical metrics, including AUROC and Fisher's Ratio. This observation was particularly pronounced in the context of the pain test, where SKNA features exhibited higher correlation coefficients with self-reported VAS pain ratings compared to EDA.

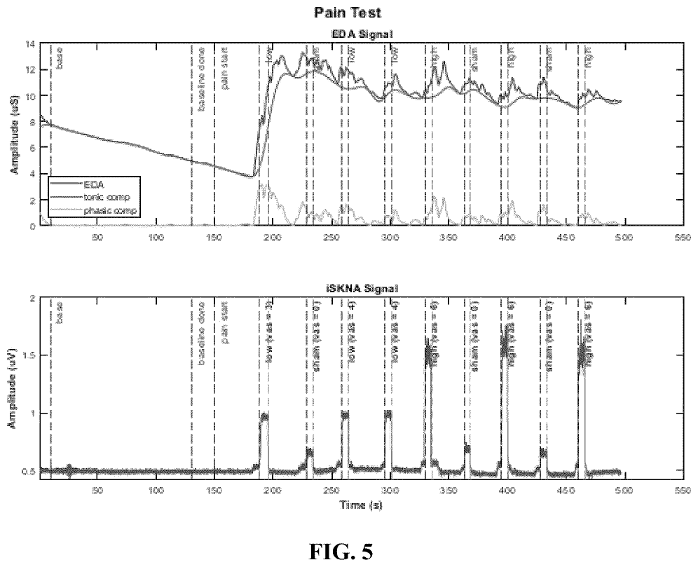

The thermal grill pain test involves the action of moving the hand above the grills before actual contact (which is indicated by vertical dotted lines in FIG. 5). Factors including stress, vocal instructions from the experiment instructor guiding subjects on when to move their hands and touch the grills, and the physical movement, all introduce artifacts that greatly reduce the specificity of EDA responses. As shown in FIG. 5, iSKNA signals are relatively less sensitive to motion artifacts in comparison, allowing for distinct observation of the bursts and obtaining more reliable data.

In the VM test, it was observed that iSKNA and EDAphasic signals exhibited a significant correlation after synchronizing the onset of EDAphasic which was initially delayed. This correlation suggests that these signals respond similarly to the same stimuli.

Moreover, SKNA bursts showed an abrupt onset immediately upon the presentation of stimuli and concluded rapidly upon stimulus removal. These sharp and concise onsets and ending patterns contrast with the slower build-up and decay of EDA bursts. The results suggest the potential of SKNA as a reliable marker for assessing pain intensity with accurate onset and ending detection of pain, a critical factor in the management and treatment of patients who have communication issues. Hence, SKNA may be especially useful for rapidly changing scenarios or when differentiation between repetitive stimuli is crucial.

To see the impact of muscle movement on SKNA signals, ECG signals were collected from ECG electrodes on both inner wrists (channel 1) and on the top left side of the chest and below the right rib (channel 2). Specifically, in the VM test, unavoidable chest and stomach movements can occur, while in the pain test, subjects were asked to move their left hand. Both channels of SKNA demonstrated consistent activity, underlining the robustness of this measure in capturing SNS activity. To ensure data quality and consistency, the channels that demonstrated the best signal quality were selected. Nonetheless, this highlights the importance of standardizing equipment and calibration procedures in future studies to enhance comparability and reliability across different measurements.

Regarding the Stroop test, some of the participants, having previously taken part in similar experiments in the lab, reported feeling less stressed than expected, possibly due to their familiarity with the experimental situation. In addition, these participants were asked to remain silent and perform the test in their minds to prevent any potential artifacts. Consequently, both EDA and SKNA exhibited minimal activation. Channel 2 of SKNA had to be excluded due to weak stimulations in both EDA and SKNA signals. Nonetheless, from channel 1 of SKNA data, HaSKNA was identified as a reliable discriminator between baseline and burst periods with significant accuracy.

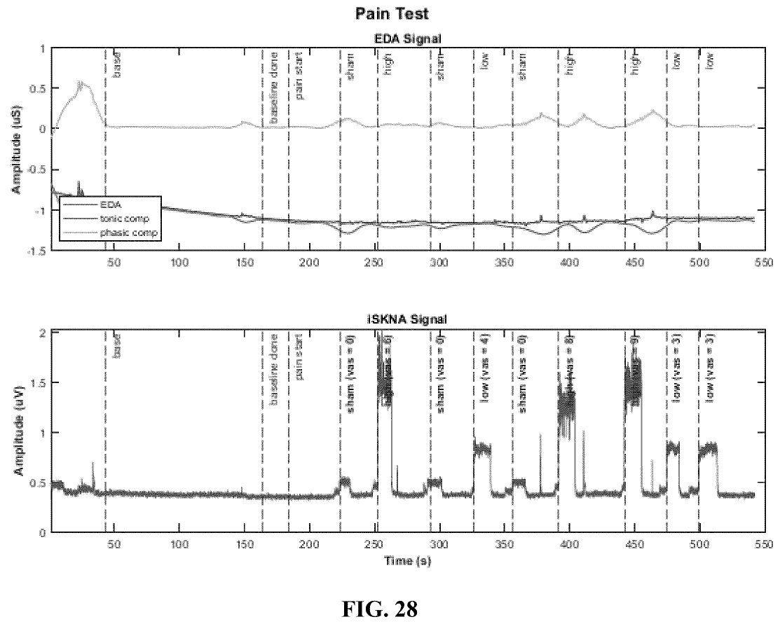

Furthermore, while one participant exhibited unreliable EDA signals, their SKNA responses remained consistent across all tests. A visual representation of the iSKNA response of this individual during the pain test is provided in FIG. 28.

The disclosure provides for potential applications of SKNA both in clinical settings where ECG signals are widely used for different purposes and in wearable devices, thereby obviating the necessity for an additional sensor for SNS assessment. Wearables with extended battery life can be designed to ensure continuous and accurate monitoring. For example, the ECG electrodes could be placed on the subject's wrists as they would for wearables and be configured to provide long-term and real-life data recording.

### Example 2: Predicting Central Nervous System Oxygen Toxicity Symptoms Using Electrodermal Activity

### 1. Context

Breathing elevated oxygen partial pressures (PO2) prior to or during diving with self-contained-underwater-breathing-apparatus (SCUBA) increases the risk of developing central nervous system oxygen toxicity (CNS-OT), which could impair performance or result in seizure and subsequent drowning. This disclosure provides a means to investigate the dynamics of electrodermal activity (EDA) while breathing elevated PO2 in the hyperbaric environment (HBO2) as a means to predict impending CNS-OT. To this end, machine learning was used to automatically detect and predict the onset of symptoms associated with CNS-OT in humans by using features derived from EDA in both time and frequency domains.

### 2. Experimental Data Set

The dataset consists of simultaneously collected EDA and electrocardiogram (ECG) from twenty-six subjects. While every subject was expected to complete two exposures to HBO2, only twenty-three subjects completed both exposures, and three subjects completed one exposure. Thus, a total of forty-nine exposures were considered.

During the experiment, subjects were immersed in 28±1° C. water up to the shoulders, breathing 100% O2 at 35 feet of seawater (oxygen partial pressure 2.06 ATA). Additionally, subjects exercised on an underwater cycle ergometer at approximately 100 W output and performed Multi-Attribute Task Battery-II (MATB-II) cognitive tests by NASA. The exposure lasted for a maximum duration of 120 minutes or until symptoms of CNS-OT were observed. For safety purposes, subjects were seated in water in a head-out position to avoid head submersion in the event of convulsion or loss of consciousness.

EDA and ECG signals were collected simultaneously at a sampling frequency of 100 Hz. EDA was collected using galvanic skin response module FF116 (ADInstruments, Sydney, Australia) while ECG signals were collected using Ag/AgCl electrodes and a Hewlett-Packard ECG monitor (Palo Alto, CA, USA).

Each exposure was annotated by four independent experts as “definite” CNS-OT, “probable” CNS-OT, or “non-CNS-OT” which is when subjects either did not exhibit any symptoms or their symptoms were concluded by experts to be not associated with CNS-OT. Finally, a majority voting poll was used to create the final adjudication. In the case of 50/50 decision splits (two annotations as “definite” and two as “probable”), the exposure was labeled as “probable.” Hence, there are eighteen “Definite CNS-OT,” thirteen “Probable,” and eighteen “non-CNS-OT” labeled exposures.

### 3. EDA Signal Preprocessing

Like most biosignals, EDA can be affected by occasional motion artifacts (MA), which may result in lower specificity for detecting CNS-OT. For more reliable results, MA should be removed from the analysis since MA can be misinterpreted as peaks corresponding to the presence of CNS-OT. Therefore, annotations from three independent EDA experts were used and a majority voting scheme was applied to separate segments with MA from clean segments. A median filter (one second window) and a low pass finite impulse response filter with a cut-off-frequency of 1 Hz were applied to remove high frequency content of the signal.

### 4. Time-Varying Spectral Analysis of EDA: TVSymp

TVSymp, which uses a time-varying spectral analysis approach, has been successfully applied in diverse applications involving stress, pain, fatigue, and dehydration detection as these cases all involve increased sympathetic innervation. TVSymp has been found to be less intra-subject variant and more sensitive when compared to the time-domain measures of EDA (i.e., tonic and phasic components).

As discussed in the previous example, TVSymp calculation involves decomposition of the EDA signal into multiple frequency bands using a high-resolution time-frequency decomposition called variable frequency complex demodulation (VFCDM). VFCDM provides both high time and frequency resolution, while retaining accurate amplitude distribution of the signal. This method then uses only the frequency bands within the range of 0.08-0.25 Hz to reconstruct the signal. Finally, an envelope of the instantaneous amplitude of the signal is computed using the Hilbert transform. This sequence of computations is described using the following equations.

The original EDA signal y (t) can be expressed as a summation of N(=12 in this case) different VFCDM components as follows:

$$y ( t ) = {Σ}_{i = 1}^{N} {C}_{i} ( t ) \qquad ( 2 )$$

Where Cᵢ represents the ith VFCDM component. Considering a sampling frequency of 2 Hz and 12 equally divided sub bands, components 2-3 contain the frequency range 0.08-0.25 Hz. Thus, the EDA signal y′ (t) is reconstructed by summing components 2 and 3 of the VFCDM decomposition:

$${y}^{′} ( t ) = {C}_{2} ( t ) + {C}_{3} ( t ) \qquad ( 3 )$$

The envelope of the signal y′(t) is then computed using the Hilbert transform. An analytic signal, A(t) of the y′(t), can be expressed as follows:

$$A ( t ) = {y}^{′} ( t ) + {jY}^{′} ( t ) \qquad ( 4 )$$

Where Y′ (t) is the Hilbert transform of the original signal y′ (t) which can be obtained by the following equation:

$${Y}^{′} ( t ) = \frac{1}{π} P {\int }_{- ∞}^{∞} {y}^{′} ( τ ) / ( t - τ ) d τ \qquad ( 5 )$$

Where P refers to the Cauchy principal value.

The instantaneous amplitude and phase of the analytic signal A (t) can be obtained as below.

$$a ( t ) = {[ {y}^{′2} ( t ) + {Y}^{′2} ( t ) ]}^{1 / 2} \qquad ( 6 )$$

$$θ ( t ) = arctan ( {Y}^{′} ( t ) / {y}^{′} ( t ) ) \qquad ( 7 )$$

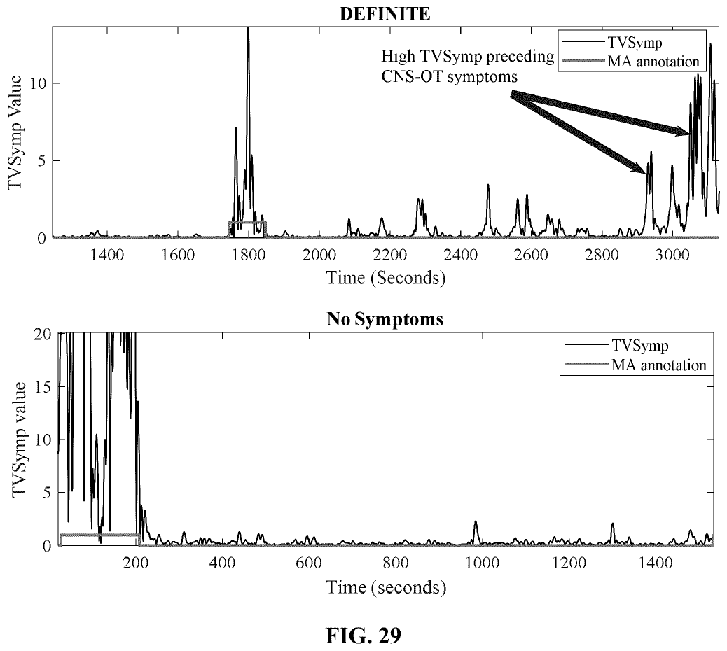

The instantaneous amplitude a (t) is considered the TVSymp time series. FIG. 29 shows the representative TVSymp time series for ‘CNS-OT’ and ‘No Symptom’ subjects. As shown in FIG. 29, preceding CNS-OT symptoms, there are significant increases in TVSymp amplitudes. Annotations for MA are also plotted (thick red rectangular box) to designate large TVSymp amplitudes due to the MA. For non-CNS-OT there is no significant sudden rise in TVSymp values as compared to the CNS-OT case.

### 5. Data Preparation for Machine Learning

For more accurate and automated prediction of seizures, the entire data recording was analyzed to identify the earliest possible onset of the sudden and large increases in TVSymp amplitudes prior to the adjudication of when CNS-OT related symptoms occurred by the experts.

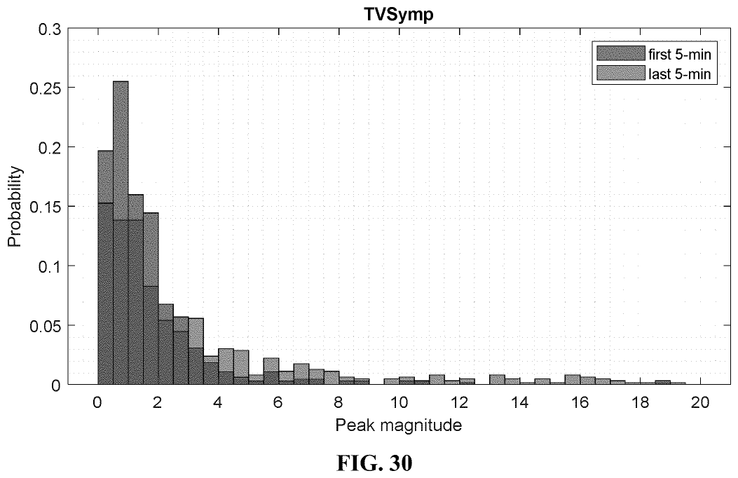

Based on the observations, TVSymp amplitudes increase significantly preceding CNS-OT related symptoms. Moreover, during the first five minutes, no seizure-related activities were observed, as there were no sudden and large increases in TVSymp amplitudes. This observation is buttressed by the histogram shown in FIG. 30, which shows TVSymp amplitudes in both the last (for those defined as ‘CNS-OT’ subjects) and first five minutes of EDA. For the first five minutes, all TVSymp amplitudes are less than approximately 4. However, in the last five minutes of the recordings (for CNS-OT subjects only), TVSymp amplitudes far exceed 4, and in some cases, they are as large as 19 when symptoms of CNS-OT tend to occur.

### 6. Feature Extraction

Four unique features were computed from each of the SCRs, including peak TVSymp amplitude, rising and falling slopes of the SCRs, and width of the SCRs. The features are described in the below table.

| Feature Name | Description |
|---|---|
| TVSymp peak | Peak amplitude of SCR TVSymp value |
| Rising slopes | $\frac{Ypeak - {Y}_{trough 1}}{{t}_{peak} - {t}_{trough 1}}$ |
| Falling slopes | $\frac{Ypeak - {Y}_{trough 2}}{{t}_{trough 2} - {t}_{peak}}$ |
| SCR width | t_{trough2} − t_{trough1} |

Trough1 and trough2 refer to the time points immediately before and after each SCR.

### 7. Machine Learning

Multiple machine learning classifiers were examined, including linear discriminant analysis (LDA), logistic regression, linear support vector machine (SVM), decision tree, random forests, and gradient boosting classifiers. Since the dataset is limited, a subject-independent leave-one-subject-out (LOSO) validation strategy was used to evaluate the machine learning models. Sensitivity and specificity were computed by averaging the predictions from each test fold.

After annotation, a total of 217 samples were related to CNS-OT and 9100 samples were not related to CNS-OT. Thus, the dataset is considered imbalanced with more negative samples. Therefore, to avoid biased training, a synthetic minority oversampling technique (SMOTE) was used to oversample the examples in the minority class.

For each fold of the LOSO validation, the hyper parameters of the classifiers were optimized using a grid-search cross-validation technique with group K-fold (k=5) cross validation on the training data. For linear SVM, the parameter C was varied from 0.01 to 1000 using multiples of 100. For random forests and gradient boosting classifiers, the maximum depth of the trees was varied from 3 to 6, and the number of estimators between 40 to 100 with a step size of 20.

To train the machine learning algorithms, only the first and last five minutes of the data were used, as supported by the histogram plot shown in FIG. 30. However, note that for validation data from the entire exposure was considered to assess how early the model detects symptoms associated with CNS-OT.

### 8. Experimental Results

For evaluating the machine learning models, a LOSO validation strategy was used and compared the ML models in terms of sensitivity and specificity, which are defined as:

$$Sensitivity = \frac{T P}{T P + F N} \qquad ( 8 )$$

$$Specificity = \frac{T N}{T N + F P} \qquad ( 9 )$$

Where, TP, TN, FP, and FN represent the number of true positive, true negative, false positive, and false negative samples, respectively. In this case, the positive class represents SCRs related to CNS-OT symptoms and the negative class represents no symptoms associated with CNS-OT.

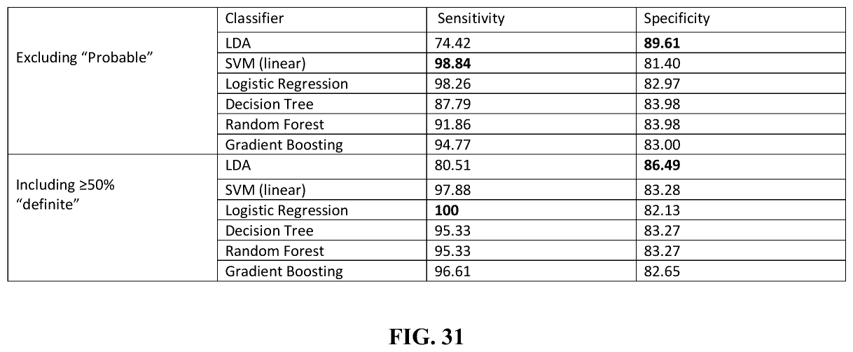

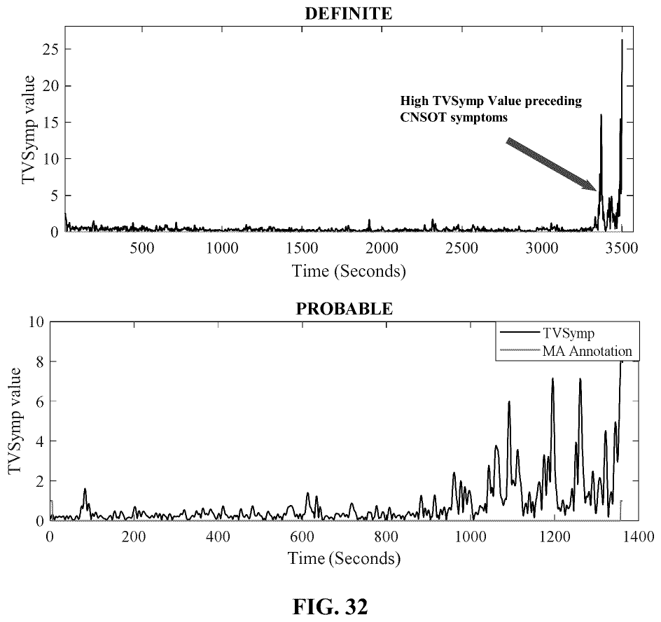

Each experimental session was annotated by four independent experts, and majority voting was considered for the final adjudicated labeling. However, for some symptomatic cases, half of the experts labeled the symptoms as “Definite CNS-OT” and the other half were unsure; hence, these cases were labeled as “Probable.” Initially, all the probable cases were excluded; however, most of them had similar EDA signatures as “Definite CNS-OT.” Given that there are limited data samples for CNS-OT, additional positive cases of CNS-OT are beneficial for better training of machine learning models. Hence, cases with “Probable” CNS-OT were included in the CNS-OT cases when at least two experts (≥50%) annotated them as “Definite CNS-OT.” This led to an additional seven definite CNS-OT cases. However, results are reported by including and excluding probable cases, as shown in FIG. 31.

FIG. 31 summarizes the machine learning results on predicting CNS-OT associated symptoms. Six different machine learning models were considered, as listed in the table. The figure shows results for both regular labeling without including “Probable” symptoms and including ≥50% “Probable” CNS-OT symptoms. Almost all of the machine learning models provided improved performance after including the ≥50% “Probable” subjects.

The linear SVM showed the maximum sensitivity of 98.84% with a good specificity of 81.40%. However, the performance of the linear SVM did not improve significantly after adding probable cases. Even though LDA provided high specificity, the sensitivity was much lower than for the other classifiers. The logistic regression classifier provided more consistent results both before and after including ≥50% “Probable” data; for the latter case, it showed 100% sensitivity with 82.13% specificity. The performance of the tree-based algorithms such as the decision tree, random forests, and gradient boosting classifiers, were comparable; all of them showed improved results after including probable cases of CNS-OT.

The fact that the performance of the machine classifier improved after including probable cases can be visualized from EDA characteristics. For example, FIG. 7 shows TVSymp series for a representative “definite” CNS-OT case and a “Probable” case. As shown in FIG. 7, both cases showed a sudden and significant rise in the TVSymp signal, which is the key signature for CNS-OT symptoms, according to the observations, albeit the amplitudes in the definite CNS-OT case were much greater than in the probable CNS-OT case. Given the limited number of definite CNS-OT cases, and the fact that probable CNS-OT cases have similar significant increases in amplitudes of TVSymp values compared to baseline, the decision to include more data from training and testing is justified and consequently led to better classification results.

### 9. Predictive Quality

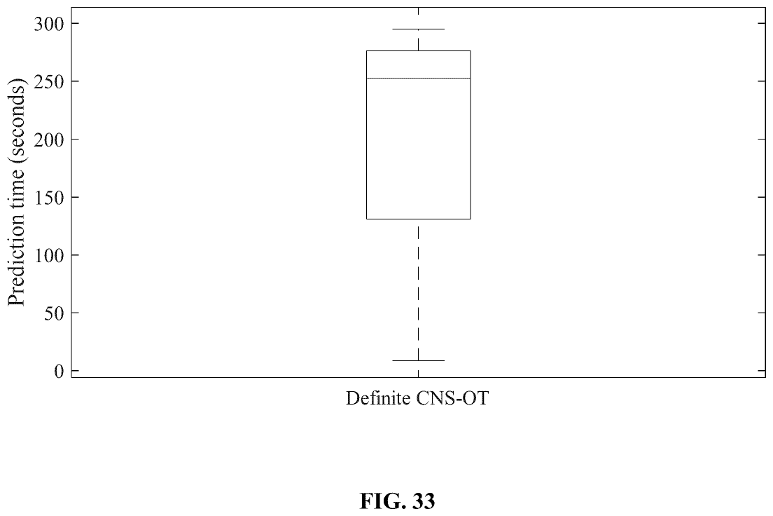

The prediction time of CNS-OT symptoms was computed by considering the first significant increase in TVSymp detected as CNS-OT. The distribution of prediction time is shown in FIG. 33. As shown in the figure, the median prediction time (indicated by the red line) is slightly above 250 seconds, while the 95% confidence interval is 199±98. This suggests that prediction of CNS-OT symptoms can be, on average, 200 seconds preceding the symptoms, which is greater than 3 minutes.

### 10. Discussion

The performance of the machine learning models showed promising results. For ethical and safety reasons for humans, the partial pressure of oxygen in this experiment was not increased beyond 2 ATA, and the subjects were removed from the experimental chamber as soon as mild symptoms associated with CNS-OT appeared. Thus, prediction was based on the presence of symptoms associated with CNS-OT rather than directing predicting seizures. Since symptoms related to CNS-OT are precursors to seizures, it is reasonable to expect that the prediction time would be even longer before actual seizures. The average prediction time was greater than three minutes before symptoms associated with CNS-OT. Approximately three minutes of warning time is sufficient for preparation of countermeasures such as alerting dive buddies and alerting the diver of impending seizures. SKNA signals may also be used for seizure detection in divers.

### 11. Techniques to Reduce False Positives and Negatives of EDA Responses.

The following disclosed techniques for processing SKNA and EDA signals can be used to reduce false positives and negatives of EDA responses. EDA consists of a tonic component, referred to as skin conductance level (SCL), and a phasic component, known as skin conductance response (SCR). SCR captures the rapid dynamics elicited by startle-like stimuli, superimposed onto the low-frequency SCL. SparsEDA and cvxEDA represent two methodologies for decomposing EDA into SCR and SCL, extracting the underlying nerve activity drivers. In both approaches, SCR is modeled as the result of the convolution between a sparse nonnegative sudomotor nerve activity phasic driver (dₚ (t)) and a biexponential impulse response function (r(t)) (Eqs. 10 and 11).

$$s ( t ) = {s}_{p} ( t ) + {s}_{l} ( t ) = {d}_{p} ( t ) * r ( t ) + {s}_{l} ( t ) , \qquad ( 10 )$$

$$r ( t ) = {e}^{- \frac{t}{{τ}_{2}}} - {e}^{\frac{t}{{τ}_{1}}} , for t \geq 0 \qquad ( 11 )$$

where s (t) is the original signal, sₗ(t) corresponds to SCL, and τ₂ and τ₁ are the time constants.

The other approach, cvxEDA, solves the problem through a convex optimization approach based on cubic B-splines for the SCR component and an autoregressive moving average model for the sudomotor SNS innervation. The output of cvxEDA is not entirely sparse, with values near zero but not precisely equal to zero. Post-processing steps are necessary to attain truly sparse drivers. SparsEDA reformulates Eq. 10 as a sparse recovery problem for the joint estimation of the SCR and SCL through the minimization of the mean squared error (MSE) between the available signal and the reconstructed one, effectively producing sparse drivers.

The biexponential model presented in Eq. 11 has been used to model the effects of individual nerve impulses on synaptic activation of the neuronal membrane. Nevertheless, SCR, due to the hydrodynamic properties of sweat pores, is a more intricate process than the firing of a single synapse and entails a prolonged response. In contrast, SKNA provides a more direct measure of nerve activity, intimately tied to the discharge activity of the stellate ganglion.

In light of this information, along with the previously established high correlation coefficient between SKNA and EDA responses noted further above, the SparsEDA technique for extracting SKNA drivers was employed. Consequently, using the sparse drivers, the capability and accuracy of SKNA and EDA signals recorded simultaneously for detecting neuronal bursts in response to sympathetic stimuli was investigated, including VM and pain induced by thermal grills in the experimental set-up described above. The EDA sensors were attached to the index and middle fingers of the right hand, recording data through the GSR Amp. Additionally, Bio Amp collected ECG signals at 10 kHz sampling frequency using Ag/Cl electrodes for lead I on the wrists with the ground electrode on the left abdomen.

Preprocessing—To obtain SKNA, bandpass filtering with cut-off frequencies of 500-1000 Hz and 1700-2000 Hz was applied to the ECG signals recorded during the VM and pain experiments, respectively. The former frequency range was used and the latter is due to an applied modification in order to get rid of an electric noise artifact from the motors pumping water through the tubes in the pain experiment. To get a smoother version of the SKNA signal, the SKNA signal was rectified and the integral area calculated, which is referred to as iSKNA. To this end, a moving average filter with a symmetric window of 0.1 second was used. The filter had overlaps equal to half the size of the window at both sides.

Cubic spline interpolation to downsample both iSKNA and EDA signals from 10 kHz to 4 Hz prior to driver extraction was used.

Driver Extraction And Baseline Removal—The SparsEDA technique was used to calculate the EDA and SKNA drivers, configuring the parameters associated with SparsEDA as follows: ϵ=10\hat{ }(−4) and Kₘₐₓ=40, Tₘᵢₙ=1.25, and ρ=0.025. The first two parameters serve as stopping criteria for the least angle regression (LARS) algorithm, a greedy method was employed to solve the LASSO (least absolute shrinkage and selection operator) transformation of the original sparse recovery problem. The Kₘₐₓ parameter determines the maximum number of iterations, while the residual of the signal reconstruction is constrained to be less than a predefined value using E. Tₘᵢₙ and p are part of the postprocessing stage of the method. Namely, the algorithm rejects adding any new driver to the set of drivers if it is within Tₘᵢₙ seconds of an existing driver, and the algorithm discards the accepted drivers whose L₀ norm is smaller than the threshold determined by the parameter p.

To extract SCR, SCL was subtracted from EDA signals. Similarly, SKNA response was obtained by subtracting the non-sparse output of the algorithm from the iSKNA signal. This modified version of iSKNA is designated as SKNAR, which is a modified version of iSKNA devoid of the subject-specific baseline variations.

Additionally, the cvxEDA algorithm was employed to decompose the z-scored EDA signal (i.e. [symbol] EDA [symbol] (z-scored)=(EDA−μ_{EDA})/σ__{EDA}, where ρ__{EDA} and σ__{EDA} represent the mean and standard deviation of the EDA signal) into its phasic (SCR) and tonic (SCL) components and extract phasic drivers. Initially, both the phasic component and the drivers were scaled by the standard deviation of the EDA. This algorithm generates a driver for each sample in the signal. Therefore, a post-processing technique was implemented to sparsify the extracted drivers.

A threshold of 0.05 μS was applied. This value is a heuristic for delineating a significant SCR. Specifically, whenever a driver's amplitude surpasses a predefined threshold and crosses over its former driver's amplitude that falls below the threshold, it is designated as a significant driver and flagged accordingly. Any other drivers failing to meet these criteria are disregarded. This process captures the concept of zero crossing, where the amplitude transitions from below to above a certain level, indicating a significant change or event. Within the accepted drivers, the local maxima with a minimum prominence of 2 was identified. The prominence of a local maximum assesses its distinctiveness in terms of height and relative position among other peaks. It is determined by extending a horizontal line from the peak, identifying outer endpoints, finding the lowest valleys in the left and right intervals, and measuring the vertical distance from the larger of these two valleys to the peak.

Segmentation And Burst Detection-Before conducting the analysis, segments of the data that were corrupted were visually excluded. Additionally, sham touches from the subsequent analysis were also excluded.

The annotated labels were considered as the ground truth for stimulus presence. Investigating 10-second segments following each label, with an additional one second preceding the label to account for potential annotation errors, instances with detected drivers within this window as “hits” and others as “misses” were classified. The hit rate was then calculated as the number of detected bursts divided by the total number of annotated stimulations (Eq. 12).

$$Hit Rate = \frac{Detected Stimulations}{Total Stimulations} \qquad ( 12 )$$

The initial driver within this window was identified as the driver for the stimulus. Upon detecting a “hit,” an examination was conducted in a subsequent 20-second window after the largest peak in a 5-second window following the first driver. The response (SCR or SKNAR) was followed to see when or whether it fell below 0.1 μS. This threshold value was chosen heuristically. If such a decline was observed, a “recovery” was noted. The recovery rate was determined by the ratio of instances where the response returns to 0.1 μS to the total number of detected stimulations, as per Eq. 13.

$$Recovery Rate = \frac{Instances of Response < 0.1}{D e t ected Stimulations} \qquad ( 13 )$$

Any additional drivers identified outside the 1+10-second specified windows in the control stage and interstimulus were classified as false alarms. The two-sample t-test was used to compare the number of false alarms generated by SKNA, cvxEDA, and SparsEDA in pairs, aiming to determine if they exhibit significant differences at a 5% significance level. If the standard deviation of one group exceeded twice that of the other in any comparison, the unequal variance test was conducted. When assuming equal variances, the statistic follows Student's t-distribution. Otherwise, an approximate t-distribution was used, with degrees of freedom estimated via Satterthwaite's approximation, known as Welch's t-test.

Temporal Features—The stimulus-to-response time (latency) of the estimated driver corresponding to each stimulus based on the associated annotated label was calculated. Additionally, \ the root mean squared error (RMSE) of the driver's time in relation to the time of the label was determined, as expressed in Eq. 14.

$$R M S E = \sqrt{{\sum }_{i = 1}^{n} \frac{{( {\hat{y}}_{i} - {y}_{i} )}^{2}}{n}} , \qquad ( 14 )$$

where n, y\hat{ }, and y represent the total number of detected stimulations, the driver's time, and the annotated label time, respectively. Ultimately, the recovery time to baseline (response duration) as the duration from the driver initiation to the point where the response falls below 0.1 μS was computed.

Finally, the two-sample t-test was employed to compare the stimulus-to-response time and the recovery time to baseline for each of the three time-series (SKNA, SparsEDA, cvxEDA).

Results—In the VM experiment, occasional false alarms are observed in the SparsEDA representation, incorrectly assigning drivers during the control stage and interstimulus intervals. Additionally, a missed burst, specifically the initial stimulation, is evident. The cvxEDA algorithm demonstrates accurate identification of all stimulation bursts. However, in contrast to the accurate alignment of the SKNA drivers with the labeled annotations, SCRs from both EDA decomposition algorithms (SparsEDA and cvxEDA) exhibit a noticeable latency in response time.

### Practical Applications

The above disclosure regarding the SKNA signals and the EDA signals and the use of machine-learning may be applied to a variety of practical applications, several of which are discussed below. The practical applications may use the SKNA signals and the EDA signals either individually or in combination. When used in combination, results obtained using one type of signal may be used to validate results obtained from the other type of signal.

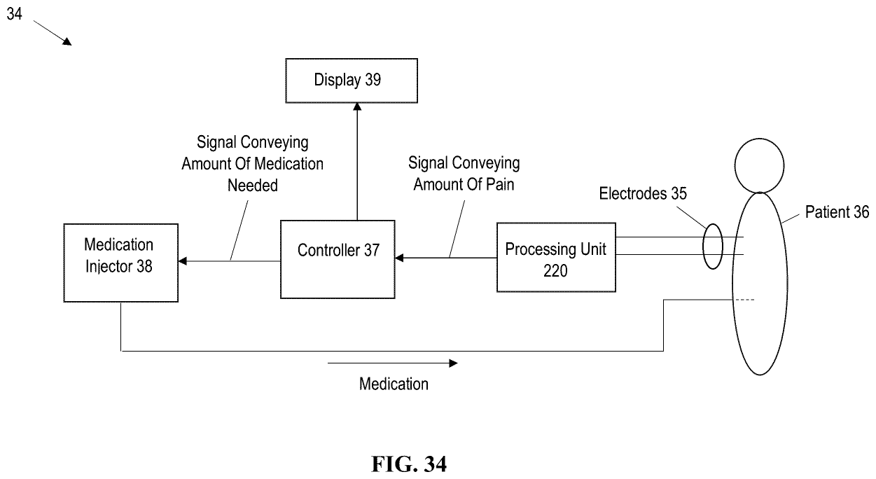

In a first example, a pain alleviation system uses the SKNA signals and/or the EDA signals to measure an amount of pain a patient may be enduring in an uncommunicative state using the disclosed machine-learning. FIG. 34 illustrates a pain alleviation system 34. The pain alleviation system 34 includes electrodes 35 for obtaining ECG signals from which SKNA signals are derived and/or EDA signals from a patient 36. The ECG signals and/or the EDA signals are received by the processing unit 220. The processing unit 220 is configured to derive the SKNA signals from the ECG signals. The processing unit 220 is also configured to apply an algorithm programmed by the machine-learning as disclosed further above to estimate an amount of pain (generally a quantitative number) being endured by the patient 36. The processing unit 220 is further configured to transmit a signal conveying the amount of pain to a controller 37. The controller 37 is configured to transmit a medication control signal to a medication injector 38. Based on the amount of pain, the controller 37 determines an amount of pain alleviating medication required by the patient with the amount of pain alleviating medication being conveyed by the medication control signal. The required amount of pain alleviating medication may be determined by a known algorithm or look-up table in non-limiting embodiments. The medication injector 38 is configured to inject the required amount of pain alleviating medication into the patient 36 generally with an intravenous (IV) needle inserted in the patient 36. The medication injector 38 may include an actuator coupled to a variable displacement pump (all not shown) that can inject a selected amount of medication. It can be appreciated that the components in this configuration provide feedback control. As such, controller 37 may implement proportional, integral, and/or derivative control among other control strategies. Alternatively or in addition, the controller 37 may be configured to output a signal to a display 39 indicating the amount of pain alleviating medication required by the patient 36 that may be administered by an attending nurse.

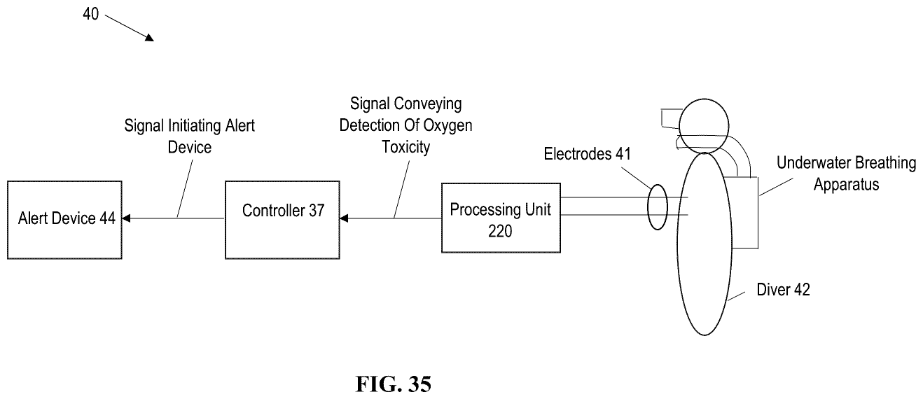

In a second example, an oxygen-toxicity detection system uses the SKNA signals and/or the EDA signals to detect on-coming oxygen-toxicity that a diver using self-contained-underwater-breathing-apparatus (SCUBA) may experience in a short amount of time. The SCUBA may be open-circuit compressed gas tanks or a closed or semi-closed circuit rebreather. From characteristics of the SKNA signals and/or the EDA signals, the oxygen-toxicity detection system detects and alerts the diver to on-coming oxygen toxicity using the disclosed machine-learning. FIG. 35 illustrates an oxygen-toxicity detection system 40. The oxygen-toxicity detection system 40 includes electrodes 41 applied to a diver 42 for obtaining ECG signals from which SKNA signals are derived and/or EDA signals from the diver 42. The ECG signals and/or the EDA signals are received by the processing unit 220. The processing unit 220 is configured to derive the SKNA signals from the ECG signals. The processing unit 220 is also configured to apply an algorithm programmed by the machine-learning as disclosed further above to detect on-coming oxygen toxicity the diver 42 may soon experience. In one or more embodiments, the detection is a binary event in which detection is based on SNS activity exceeding a threshold level. The oxygen-toxicity detection system 40 includes an alert device 43, which emits energy to alert the diver 42. Non-limiting embodiments of the alert device 44 include a light, which may flash, positioned to be viewed by the diver 42, an acoustic speaker, and a vibration emitter attached to the diver 42 so that the diver 42 may sense emitted vibration when an alert signal is emitted. It can be appreciated that the above components are generally watertight and pressure resistant for being operational in an underwater environment. Alternatively, the components may be disposed in a watertight and pressure resistant housing to provide isolation from the hazards of the underwater environment.

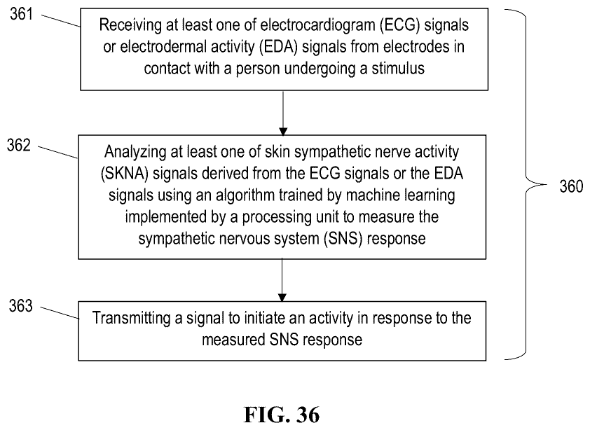

FIG. 36 illustrates a flow chart for a method 360 for performing an activity based on a sympathetic nervous system (SNS) response to a stimulus. Block 361 calls for receiving at least one of electrocardiogram (ECG) signals or electrodermal activity (EDA) signals from electrodes in contact with a person undergoing a stimulus. Block 362 calls for analyzing at least one of skin sympathetic nerve activity (SKNA) signals derived from the ECG signals or the EDA signals using an algorithm trained by machine learning implemented by a processing unit to measure the SNS response. Block 363 calls for transmitting a signal to initiate the activity in response to the SNS response. In one or more embodiments, the activity is administering a pain alleviating medication to an uncommunicative patient. In one or more embodiments, the activity is emitting an alert signal to a diver warning the diver that the diver may be shortly undergoing oxygen toxicity.

The disclosed AI models trained and implemented in such a new and different way are beyond what is achievable by pen and paper or prior techniques, removing or reducing the time-consuming and laborious—and quite often inaccurate—behavior of manual analysis. Further, techniques described herein are not those previously used in a manual process. These specific techniques, as described herein, for training and application of the AI models are an improvement in technology or technical field related to measuring SNS activity. As discussed above, the techniques described herein at least improve certain applications in which SNS activity is an indicator of a biological event. Further, the techniques described herein do not pre-empt every method of improving treatment or monopolize the basic tools of scientific or technological work.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximately”) one particular value, and/or to “about” (or “approximately”) another particular value. When such a range is expressed, another embodiment includes from the one particular value and/or to the other particular value. Similarly, when values are expressed as approximations, by use of the antecedent “about” or “approximately” it will be understood that the particular value forms another embodiment. It will be further understood that the endpoints of each of the ranges are disclosed both in relation to the other endpoint, and independently of the other endpoint.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

As used herein, the term “about” refers to a range of values of plus or minus 10% of a specified value. For example, the phrase “about 200” includes plus or minus 10% of 200, or from 180 to 220, unless clearly contradicted by context.

As used herein, “optional” or “optionally” means that the subsequently described event or circumstance may or may not occur, and that the description includes instances where said event or circumstance occurs and instances where it does not.

As used herein, the term “subject” and “patient” as used herein interchangeably refers to any living being. In some embodiments, the subject may be a human or a non-human. In some embodiments, the subject is a human. The subject or patient may be undergoing other forms of treatment.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112 (f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements, unless otherwise indicated herein or clearly contradicted by context. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

The disclosure illustratively disclosed herein may be practiced in the absence of any element which is not specifically disclosed herein.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope. Other embodiments will be apparent to those skilled in the art from consideration of the specification and practice disclosed herein. It is intended that the specification and examples be considered with only a true scope being indicated by the following claims.

What is claimed is:

1. An apparatus for performing an activity based on a sympathetic nervous system (SNS) response to a stimulus, the apparatus comprising:
an electrode configured to contact a person undergoing the stimulus;
a processing unit comprising an algorithm programmed by machine-learning and configured to: (i) receive at least one of electrocardiogram (ECG) signals or electrodermal activity (EDA) signals from the electrode; (ii) analyze at least one of skin sympathetic nerve activity (SKNA) signals derived from the ECG signals or the EDA signals to measure the SNS response and provide an SNS response measurement; and (iii) transmit an SNS response measurement signal to initiate the activity in response to the SNS response measurement; and
an activity device configured to perform the activity in response to the SNS response measurement signal.

2. The apparatus according to claim 1, wherein the SNS response is due to pain and the activity device comprises a medication injector.

3. The apparatus according to claim 1, wherein the activity device comprises a display disposed in a medical care environment and configured to receive the activity signal, wherein the activity signal comprises an amount of medication to be administered to the person.

4. The apparatus according to claim 1, wherein the SNS response is due to oxygen-toxicity and the activity device is a submersible alert device configured to emit energy to alert a diver to oxygen-toxicity going forward.

5. The apparatus according to claim 1, wherein the processing unit is further configured to at least one of: (iv) extract the SKNA signals from the ECG signals; calculate rectified SKNA (iSKNA) signals from the extracted SKNA signals; and extract features from the iSKNA signals or (v) extract a phasic component from the EDA signals.

6. The apparatus according to claim 5, wherein the processing unit is further configured to receive at least one of baseline ECG signals or baseline EDA signals indicative of no stimulus being applied to the person.

7. The apparatus according to claim 6, wherein the processing unit is further configured to: process the baseline ECG signals to extract baseline SKNA; calculate baseline rectified SKNA signals (iSKNA) from the extracted baseline SKNA; extract baseline features from the iSKNA signals; and compare the extracted baseline features to corresponding features of the stimulated ECG signals to measure the SNS response.

8. The apparatus according to claim 6, wherein the processing unit is further configured to: to process the extract a phasic component from the baseline EDA signals; extract features from the phasic component of the baseline EDA signals; and compare the extracted features corresponding to the baseline EDA signals to corresponding features of the stimulated EDA signals to measure the SNS response.

9. The apparatus according to claim 1, wherein the processing unit is further configured to train the algorithm using at least one of extracted features from training iSKNA signals or extracted features from training EDA signals.

10. The apparatus according to claim 1, wherein the processing unit is configured to measure the SNS response in real time.

11. The apparatus according to claim 1, wherein the processing unit is further configured to apply a motion artifact reduction algorithm to the ECG signals before extracting the SKNA to reduce the impact of subject movement on the SNS response measurement.

12. A non-transitory computer-readable medium storing instructions that, when executed by a processor, cause the processor to perform a method for measuring a sympathetic nervous system (SNS) response to a stimulus, the method comprising:
receiving at least one of electrocardiogram (ECG) signals or electrodermal activity (EDA) signals form the electrode;
analyzing at least one of skin sympathetic nerve activity (SKNA) signals derived from the ECG signals or the EDA signals to measure the SNS response and provide an SNS response measurement; and
transmitting an SNS response measurement signal to initiate the activity in response to the SNS response measurement.

13. The non-transitory computer-readable medium according to claim 12, wherein the method performed by the processor further comprises at least one of: (vi) extracting the SKNA signals from the ECG signals; calculating rectified SKNA (iSKNA) signals from the extracted SKNA signals; and extracting features from the iSKNA signals or (vii) extracting a phasic component from the EDA signals.

14. The non-transitory computer-readable medium according to claim 13, wherein the method performed by the processor further comprises receiving at least one of baseline ECG signals or baseline EDA signals indicative of no stimulus being applied to the person.

15. The non-transitory computer-readable medium according to claim 14, wherein the method performed by the processor further comprises extracting baseline SKNA; calculating baseline rectified SKNA signals (iSKNA) from the extracted baseline SKNA; extracting baseline features from the iSKNA signals; and comparing the extracted baseline features to corresponding features of the stimulated ECG signals to measure the SNS response.

16. The non-transitory computer-readable medium according to claim 14, wherein the method performed by the processor further comprises extracting a phasic component from the baseline EDA signals; extracting features from the phasic component of the baseline EDA signals; and comparing the extracted features corresponding to the baseline EDA signals to corresponding features of the stimulated EDA signals to measure the SNS response.

17. A method for performing an activity based on a sympathetic nervous system (SNS) response to a stimulus, the method comprising:
receiving at least one of electrocardiogram (ECG) signals or electrodermal activity (EDA) signals from an electrode;
analyzing at least one of skin sympathetic nerve activity (SKNA) signals derived from the ECG signals or the EDA signals to measure the SNS response and provide an SNS response measurement;
transmitting an SNS response measurement signal to initiate the activity in response to the SNS response measurement; and
performing the activity using an activity device in response to the SNS response measurement signal.

18. The method according to claim 13, further comprising administering an amount of medication to a person in contact with the electrode.

19. The method according to claim 14, wherein administering comprises injecting using a medication injector.

20. The method according to claim 13, further comprising emitting energy to alert a diver to oxygen-toxicity going forward.
